(19) United States
(12) Patent Application Publication
(10) Pub. No.: US 2024/0175093 A1
(43) Pub. Date: May 30, 2024

(54) MOLECULAR SUBTYPING OF COLORECTAL LIVER METASTASES TO PERSONALIZE TREATMENT APPROACHES

(71) Applicants: The University of Chicago, Chicago, IL (US); NorthShore University HealthSystem, Evanston, IL (US)

(72) Inventors: Sean PITRODA, Chicago, IL (US); Mitchell POSNER, Chicago, IL (US); Ralph WEICHSELBAUM, Chicago, IL (US); Mark TALAMONTI, Evanston, IL (US)

(73) Assignees: The University of Chicago, Chicago, IL (US); NorthShore University HealthSystem, Evanston, IL (US)

(21) Appl. No.: 18/552,414

(22) PCT Filed: Mar. 25, 2022
(86) PCT No.: PCT/US22/21978

§ 371 Date: Sep. 25, 2023

Related U.S. Application Data
(60) Provisional application No. 63/166,155, filed on Mar. 25, 2021.

(51) Int. Cl. C12Q 1/6886; C12Q 1/686; G16B 25/10; G16H 50/20

(52) U.S. Cl. CPC C12Q 1/6886; C12Q 1/686; G16B 25/10; G16H 50/20; C12Q 2600/106; C12Q 2600/112; C12Q 2600/118; C12Q 2600/158; C12Q 2600/178

(57) ABSTRACT

Methods, assays, and compositions for identifying molecular subtypes of metastatic cancer are disclosed. Methods include determining expression levels of genes and/or miRNAs in a sample of metastatic tissue and identifying the molecular subtype of the metastasis based on the determined expression levels using a neural network-based classifier. Methods may further include providing a prognosis and making treatment decision based on the molecular subtype of the metastasis.

### CROSS-REFERENCE TO RELATED APPLICATIONS

This application claims the benefit of priority of U.S. Provisional Patent Application No. 63/166,155 filed Mar. 25, 2021, which is hereby incorporated by reference in its entirety.

### BACKGROUND

### 1. Field of the Invention

The current disclosure relates generally to molecular biology and medicine. Particularly it concerns the field of oncology. More particularly, the disclosure relates to methods and compositions involving diagnosis and treatment of metastatic cancer, including metastatic colorectal cancer.

### 2. Technical Background

Metastases are the leading cause of cancer-related deaths and are frequently widely disseminated, which has led to the prevailing view that metastases are always widespread. The oligometastasis hypothesis, in contrast, suggests that metastatic spread is a spectrum of virulence where some metastases are limited both in number and organ involvement and potentially curable with surgical resection or other loco-regional therapies^{1,2}. This paradigm is in stark contrast to the outcomes of patients with solid tumors where widespread metastases are largely fatal despite recent advances in systemic therapy. To date, the oligometastasis concept has been challenged, in large part, due to the lack of supporting molecular data to identify metastases associated with restricted spread^{3,4}.

Limited metastasis is relatively common. Data from clinical trials and single institution analyses of lung, breast, colorectal, prostate and renal cancers suggest that as many as 40-60% of patients with metastasis present with or develop limited disease⁵⁻⁸. Patients with limited liver metastases from colorectal cancer (CRC) have been consistently demonstrated to achieve prolonged survival after hepatic resection^{9,10} and provide an opportunity to investigate the molecular basis for oligometastasis. While there have been extensive investigations into the molecular subtypes of primary human cancers, little is known regarding molecular subtypes of metastasis and their relation to clinical outcomes.

Expression signatures based on mRNA or miRNA expression levels in metastatic tissue have been developed. Pitroda et al., “Integrated molecular subtyping defines a curable oligometastatic state in colorectal liver metastasis,” Nature Communications 9:1793 (2018), which is hereby incorporated by reference, describes identification of three subtypes of liver metastases from colorectal cancer primary tumors using expression levels of mRNAs and miRNAs in metastatic tissue (see also PCT Publication WO2019/204576 to Pitroda et al., which is hereby incorporated by reference). Classification of the subtypes depended on mRNA or miRNA signatures requiring analysis of approximately 50 to 200 miRNAs or mRNAs, and it was only possible to classify patients into one of two groups (one SNF2 group, and one SNF1+SNF3 group). A validated classification process that requires fewer expression level inputs and accurately identifies all three metastatic molecular subtypes would help to improve the efficiency and reliability of identifying molecular subtypes of metastases.

There remains a need for robust, externally validated methods of identifying molecular subtypes of metastatic cancer that are predictive of clinical outcome and that can inform treatment decisions and prognosis for metastatic cancer.

### SUMMARY

The inventors have discovered and validated a classification process that identifies molecular subtypes of cancer metastases and meets the needs described above. The inventors have developed methods of diagnosis, prognosis, and treatment that use the molecular classification of metastatic tissue to identify curable metastatic cancer and otherwise guide treatment decisions. Using a multi-layer neural network analysis of gene and miRNA expression data in metastatic tissue samples, the inventors identified expression signatures that reliably classify metastatic samples into one of three subtypes—canonical, immune, and stromal—which correlate with different clinical outcomes and different treatment indications. Surprisingly, the neural network classification analysis can identify the subtype of a metastatic tissue sample based on fewer mRNA and miRNA expression levels than was possible in previously known methods. The three subtypes correlate with different clinical outcomes, and knowing the subtype of the metastasis informs treatment decisions and helps provide an accurate assessment of patient prognosis. This discovery applies in metastatic cancers beyond only colorectal liver cancer—methods disclosed herein can be used to identify molecular subtypes of other metastatic cancers and to guide prognosis and treatment decisions for patients having such cancers.

Described herein, in some aspects, is a method comprising measuring expression levels of one or more genes listed in Table 1 and/or one or more miRNAs listed in Table 2 in a sample comprising tissue from a metastasis from a primary cancer tumor. Described herein, in some aspects, is a method comprising measuring expression levels of one or more genes and/or one or more miRNAs listed in Table 6 in a sample comprising tissue from a metastasis from a primary cancer tumor. These tables list genes and miRNAs whose expression is particularly valuable in classifying molecular subtypes of metastases. In some embodiments, expression of other genes and miRNAs are also measured, including, for example, genes and miRNAs that are differentially expressed in canonical, immune, or stromal molecular subtypes of metastases. In some embodiments, expression of both genes and miRNAs are measured as part of a method disclosed herein. The methods disclosed herein can be used specifically in the context of metastatic colorectal cancer. Thus, in some embodiments, the metastasis may be a liver metastasis, and the cancer may be colorectal cancer. The metastasis that is tested may also be in other parts of the body besides the liver, including the lung, peritoneum, brain, or bone. The methods disclosed herein can also be used in the context of other metastatic cancers including, for example, liver cancer, testicular cancer, biliary cancer, ovarian cancer, urinary tract cancer, pancreatic cancer, prostate cancer, esophageal cancer, gastric cancer, head and neck cancer, cervical cancer, lung cancer, neuroendocrine cancer, kidney cancer, breast cancer, and melanoma. In some embodiments, the expression levels of the one or more genes or one or more miRNAs indicate that the metastasis has a canonical, immune, or stromal phenotype. In some embodiments, an expression signature of the one or more genes or one or more miRNAs matches an expression signature of a canonical, immune, or stromal metastatic phenotype. In some embodiments, the method further comprises calculating a clinical risk score for the patient. In some embodiments, the clinical risk score is derived from clinical characteristics of the patient, such as (1) disease-free interval between primary tumor diagnosis and development of metastasis<12 months, (2) number of liver metastases>1, (3) largest liver metastasis>5 cm, (4) lymph node-positive primary CRC, and (5) CEA>200 ng/mL. A patient with none of these features has a CRS of 0; a patient with one of these features has a CRS of 1; and so on up to a maximum CRS of 5. Clinical Risk Score (CRS) is a widely accepted prognostic tool for CRC patients undergoing liver metastasis resection^{9,12,13}.

In some embodiments, the method further comprises administering a cancer therapy to the patient. The cancer therapy may be chosen based on the gene or miRNA expression measurements, alone or in combination with the clinical risk score calculated for the patient. In some embodiments, the cancer therapy comprises a local cancer therapy. In some embodiments, the cancer therapy excludes a systemic cancer therapy. In some embodiments, the cancer therapy excludes a local therapy. In some embodiments, the cancer therapy comprises a local cancer therapy without the administration of a system cancer therapy. In some embodiments, the cancer therapy comprises an immunotherapy, which may be an immune checkpoint therapy. In some embodiments the cancer therapy comprises cetuximab or panitumumab. Any of these cancer therapies may also be excluded in certain embodiments. Combinations of these therapies may also be administered. In some embodiments, the gene or miRNA expression measurement and analysis may indicate that one or more cancer therapies would be likely to be effective or ineffective. A particular advantage of methods disclosed herein is that they allow doctors to make a treatment decision based on the molecular subtype of a metastasis. The discoveries disclosed herein indicate that some metastatic subtypes, such as immune, for example, are more likely to respond to a local therapy such as resection, radiation therapy, and the like, without the need for a systemic cancer therapy. The discoveries disclosed herein also allow doctors to identify metastatic cancer for which a local therapy may not be helpful and/or for which systemic therapies, such as DNA damaging drugs, are appropriate.

In some embodiments, the expression levels of at least, at most, or exactly 1, 2, 3, 4, 5, 6, 7, 8, 9, 10, 11, 12, 13, 14, 15, 16, 17, 18, 19, 20, 21, 22, 23, or 24 of the genes listed in Table 1 are measured, or any range derivable therein. In some embodiments, the expression levels of at least, at most, or exactly 1, 2, 3, 4, 5, 6, 7, 8, 9, 10, 11, 12, 13, 14, 15, 16, 17, 18, 19, 20, 21, 22, 23, or 24 of the genes listed in Table 1 are excluded from being measured, or any range derivable therein. In some embodiments, the expression levels of at least, at most, or exactly 1, 2, 3, 4, 5, 6, or 7 of the miRNAs listed in Table 2 are measured, or any range derivable therein. In some embodiments, the expression levels of at least, at most, or exactly 1, 2, 3, 4, 5, 6, or 7 of the miRNAs listed in Table 2 are excluded from being measured, or any range derivable therein. In some embodiments, the expression levels of one or more genes listed in Table 1 and one or more miRNAs listed in Table 2 are measured. In some embodiments, expression levels of all 24 of the genes listed in Table 1 and expression levels of all 7 of the miRNAs in Table 2 are measured. In some embodiments, the expression levels of at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 of the genes and miRNAs in Table 6 are measured. In some embodiments, expression levels of all 31 of the genes and miRNAs in Table 6 are measured. In some embodiments, the expression levels of at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 of the genes and miRNAs listed in Table 6 are excluded from being measured,

It is contemplated that expression levels of any subset of the genes or miRNAs listed in Tables 1 and 2 may be measured or may be excluded from being measured as part of a method disclosed herein. Certain subsets of these genes and miRNAs may be chosen for their greater usefulness in making classifications and differentiating between different types of metastases. A subset of genes or miRNAs that are to be examined as part of an assay to identify a sample metastasis as belonging to a particular molecular subtype may be identified by an analysis such as a nearest shrunken centroid analysis to identify subsets of genes and/or miRNAs, or a combination of genes and miRNAs, whose expression levels best characterize each subtype. Methods disclosed herein may include performing such an analysis to identify a set of genes and/or miRNAs that can provide for accurate and sensitive subtyping of individual metastases.

In some embodiments, the expression levels of one or more genes and/or one or more miRNAs are within a predetermined amount of the mean expression levels of the one or more genes or miRNAs, on a gene-by-gene and miRNA-by-miRNA basis, in metastases of a cohort of patients having canonical subtype metastases, of a cohort of patients having immune subtype metastases, of a cohort of patients having stromal subtype metastases, of a cohort of patients having an oligometastatic phenotype, of a cohort of patients who are likely to be healed without the administration of systemic cancer therapy, of a cohort of patients having a mean five-year overall survival expectation that is at least 60% or is less than 60%, or of a cohort of patients having a mean five-year disease-free survival expectation that is at least 30% or is less than 30%. The mean levels may be determined by measuring the expression levels of genes in metastases of patients in the cohort and calculating a mean expression level for each gene. In some embodiments, the patients are patients having metastatic cancer or having metastatic colorectal cancer. Classification of a metastasis may be done by comparing the measured expression levels of genes and/or miRNAs to reference expression levels of the same genes and/or miRNAs. The reference expression levels may be identified as the mean expression levels in metastases of a cohort of patients having characteristics associated with a metastatic subtype, such as a cohort having a mean five-year overall survival expectation that is at least 60% or less than 60% or a mean five-year disease-free survival expectation that is at least 30% or is less than 30%, or other characteristics of a molecular subtype, such as the characteristics of a canonical, immune, or stromal subtype described herein. The reference expression levels of such cohorts, and of any patient cohorts described herein, may be established by measuring the expression levels in metastases of at least, at most, or exactly 50, 100, 150, 200, 250, 300, 350, 400, 450, 500, 600, 700, 800, 900, or 1000 subjects in the cohort, or any range derivable therein. In some embodiments, the cohort of patients comprises a representative sample of metastatic cancer patients, including metastatic colorectal cancer patients, having a certain characteristic, such as an oligometastatic phenotype, a relatively high likelihood of being successfully treated with immune checkpoint therapy, a mean five-year overall survival expectation of at least 60% or less than 60% or a mean five-year disease-free survival expectation of at least 30% or less than 30%, or other characteristics of metastatic subtypes identified herein. If the expression levels of the genes and/or miRNAs measured in a sample metastasis are sufficiently close to the reference expression levels of a metastatic subtype, then the sample metastasis can be classified as being of that subtype. The degree of closeness in expression levels required to be classified as a match may be predetermined using a statistical analysis, including a neural network classification process. In some embodiments, the predetermined amount of closeness is within one standard deviation of the mean expression level of the reference cohort. In some embodiments, the predetermined amount is within 0.1, 0.5, 1.0, 2.0, 3.0, 4.0, 5.0, 10, 15, or 20% of the reference expression level, or any range derivable therein. In some embodiments, a sample metastasis may be classified as belonging to a molecular subtype despite the expression levels of one or more genes or miRNAs deviating from a reference expression level by a substantial amount. For instance, if a substantial number of other gene or miRNA expression levels sufficiently match the reference expression, then the sample metastasis may be classified as belonging to the subtype. A computer-based classifier programmed to perform a statistical analysis may be used to determine whether expression levels of a sufficient number of genes and/or miRNAs in a sample metastasis are sufficiently close to the reference expression levels of a particular molecular subtype to classify the sample as belonging to that subtype. The computer-based classifier program may comprise a neural network classification process or may have been derived using a neural network process.

In some embodiments, expression levels of the one or more genes or miRNAs are analyzed using a multi-layer neural network classification process. In some embodiments, the multi-layer neural network classification process includes an input layer, one or more hidden layers, and an output layer. In some embodiments, the neural network process uses expression levels of one or more of the genes listed in Table 1 and/or one or more of the miRNAs listed in Table 2 as an input layer. In some embodiments, the neural network process uses expression levels of all 24 genes listed in Table 1 and all 7 of the genes listed in Table 2 as an input layer. In some embodiments, the neural network process uses only the expression levels of genes listed in Table 1 and the miRNAs listed in Table 2 as the input layer and excludes all other genes. In some embodiments, the inputs into the input layer of the neural network process consist of expression levels of the 24 genes listed in Table 1 and the 7 miRNAs listed in Table 2. In some embodiments, the inputs into the input layer of the neural network process consist of the expression levels of 1, 2, 3, 4, 5, 6, 7, 8, 9, 10, 11, 12, 13, 14, 15, 16, 17, 18, 19, 20, 21, 22, 23, or 24 of the genes listed in Table 1 and 1, 2, 3, 4, 5, 6, or 7 of the miRNAs listed in Table 2, or any combination of these 31 expression levels, or all 31 of these expression levels. In some embodiments, the expression levels are nodes of the input layer. In some embodiments, the input layer has at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 nodes. In some embodiments, the multi-layer neural network classification process comprises 1, 2, 3, 4, 5, 6, 7, 8, 9, 10, 11, 12, 13, 14, 15, 16, 17, 18, 19, or 20 hidden layers, or any range derivable therein. In some embodiments, each hidden layer comprises one or more neurons, or nodes. In some embodiments, each hidden layer comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 neurons or nodes. In some embodiments, the classification process comprises determining the probability that the metastasis has a canonical, immune, or stromal metastatic phenotype. In some embodiments the output layer of the neural network classification process comprises an indication of the probability that the metastasis tissue sample is of a canonical, immune, or stromal molecular subtype. In some embodiments, the output layer comprises a classification of the expression level data of the input layer as indicating a canonical, an immune, or a stromal metastatic phenotype. In some embodiments, the output layer comprises three nodes, each of which indicating a probability that the metastasis is one of a canonical, immune, or stromal molecular subtype, and the metastasis is identified as being of the subtype with the highest probability. In some embodiments, the output layer consists of the three nodes. In some embodiments, the output layer comprises or consists of a first hidden layer and a second hidden layer. In some embodiments, the first hidden layer has 35 neurons and the second hidden layer has 3 neurons. In some embodiments, the first hidden layer has at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 neurons or nodes, or any range between any two of these values. In some embodiments, the second hidden layer has at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 neurons or nodes, or any range between any two of these values.

Measuring the expression of genes and/or miRNAs may be done by a variety of methods. In some embodiments, the measurement comprises performing PCR using RNA obtained from a sample of metastatic tissue as a template. The method may include the use of sets of PCR primers that are complementary to sequences of genes or miRNAs listed in Tables 1 and 2, including any subsets thereof. Measuring expression may also comprise hybridizing nucleic acids to a microarray. The microarray may include nucleic acid sequences that correspond to or are complementary to sequences of genes or miRNAs listed in Tables 1 and 2, including any subsets thereof. Methods may also include the use of nucleic acid probes that correspond to or are complementary to sequences of genes or miRNAs listed in Tables 1 and 2. Any of the primers or probes used may be labeled or modified with fluorescent labels or other moieties that allow the primers or probes to be detected. In some embodiments, measuring expression comprises performing RNA sequencing.

Also disclosed is a method of treating metastatic cancer in a patient, the method comprising administering to the patient a local cancer therapy without administering systemic cancer therapy, administering to the patient an immunotherapy, or administering to the patient cetuximab, wherein the patient has been determined to have a metastasis having expression levels of one or more genes listed in Table 1 or one and/or more miRNAs listed in Table 2 that indicate a canonical or immune metastatic phenotype based on a multi-layer neural network classification process. Embodiments of the method may use a neural network classification process having the features disclosed above. In some embodiments, the classification process uses only the 24 genes listed in Table 1 and the 7 miRNAs listed in Table 2 as inputs.

Also disclosed is a method of treating metastatic cancer in a patient, the method comprising administering to the patient a local cancer therapy without administering systemic cancer therapy or administering to the patient an immunotherapy or cetuximab, wherein the patient has been determined to have a metastasis having expression levels of one or more genes listed in Table 1 or one or more miRNAs listed in Table 2 that are within a predetermined amount of the mean expression level of the one or more genes or miRNAs in metastases of a cohort of metastatic cancer patients having a mean overall five-year survival expectation that is at least 60% or a mean five-year disease-free survival expectation that is at least 30%. In some embodiments, only the 24 genes listed in Table 1 and the 7 miRNAs listed in Table 2 are analyzed. In some embodiments, the expression levels of the one or more genes indicate a canonical or immune metastatic phenotype. In some embodiments, an expression signature of the one or more genes or one or more miRNAs matches an expression signature of a canonical or immune metastatic phenotype.

Also disclosed is a method of treating metastatic cancer in a patient, the method comprising administering to the patient a local cancer therapy without administering systemic cancer therapy, wherein the patient has been determined to have an mRNA and/or miRNA expression profile indicating canonical or immune metastatic phenotype, wherein the mRNA expression profile is determined by determining the expression of one or more genes listed in Table 1 and the miRNA expression profile is determined by determining the expression of one or more genes listed in Table 2. In some embodiments, the expression of one or more genes listed in Table 1 and one or more miRNAs listed in Table 2 are used as the input layer of a multi-layer neural network classification process. In some embodiments, the input layer consists of the 24 genes listed in Table 1 and the 7 miRNAs listed in Table 2.

Also disclosed is a method of treating cancer in a patient having a metastasis from a primary cancer tumor, the method comprising: administering to the patient an immune checkpoint therapy or administering to the patient a local cancer therapy without administering a systemic cancer therapy, wherein the patient has been identified based on expression levels of one or more mRNA and/or miRNA species in the metastasis as belonging to a group of metastatic cancer patients with one or more of the following characteristics: (a) a mean five-year overall survival expectation of at least 60%; (b) a mean five-year disease-free survival expectation of at least 30%; (c) a likelihood of experiencing metastatic recurrence after hepatic resection that is lower than the likelihood for patients outside of the group; (d) a canonical metastatic phenotype; and (e) an immune metastatic phenotype; wherein the one or more the one or more mRNA species comprise 1, 2, 3, 4, 5, 6, 7, 8, 9, 10, 11, 12, 13, 14, 15, 16, 17, 18, 19, 20, 21, 22, 23, or 24 of the genes listed in Table 1; and wherein the one or more miRNA species comprise at least 1, 2, 3, 4, 5, 6, or 7 of the miRNAs listed in Table 2. In some embodiments, the one or more mRNA species do not comprise transcripts of any genes other than those listed in Table 1, and the one or more miRNA species do not comprise any miRNAs other than the miRNAs listed in Table 2. In some embodiments, the metastasis is a liver metastasis and the cancer is colorectal cancer.

Also disclosed is a method of diagnosing a patient having a metastasis from a primary colorectal cancer tumor, the method comprising: (a) determining expression levels in the metastasis of one or more of the genes listed in Table 1 or of one or more miRNAs listed in Table 2; (b) identifying the patient as having a canonical metastatic phenotype, as having an immune metastatic phenotype, as being a responder to immune checkpoint cancer therapy, as having a five-year overall survival expectation of greater than 60%, or as having a five-year disease-free survival expectation of greater than 30% if the expression level of one or more of the genes or miRNAs is within a predetermined amount of a first reference expression level or deviates from a second reference expression level by a predetermined amount. In some embodiments, the first reference expression level represents the mean expression level in metastases of a cohort of metastatic cancer patients having a canonical metastatic phenotype, having an immune metastatic phenotype, being a responders to immune checkpoint cancer therapy, having a five-year overall survival expectation of greater than 60%, and/or having a five-year disease-free survival expectation of greater than 30%. In some embodiments, the second reference expression level represents the mean expression level in metastases of a cohort of metastatic cancer patients having a mean five-year overall survival expectation of less than 60%.

Also disclosed is a method of diagnosing and treating a patient having a metastasis from a primary colorectal cancer tumor, the method comprising: (a) measuring the expression of one or more genes listed in Table 1 or one or more miRNAs listed in Table 2 in a sample from the metastasis; (b) comparing the measured expression level of each gene or miRNA to a reference expression level for that gene or miRNA; (c) identifying the metastasis as having a canonical, immune, or stromal phenotype based on the measured expression levels; and (d) administering to the patient an appropriate therapy based on the type of metastasis identified in step (c). In some embodiments, step (a) comprises measuring the expression of at least 1, 2, 3, 4, 5, 6, 7, 8, 9, 10, 11, 12, 13, 14, 15, 16, 17, 18, 19, 20, 21, 22, 23, or 24 of the genes listed in Table 1 and/or at least 1, 2, 3, 4, 5, 6, or 7 of the miRNAs listed in Table 2. In some embodiments, step (b) comprises analyzing the expression level of each gene or miRNA using a multi-layer neural network classification system having an input layer, one or more hidden layers, and an output layer, wherein the input layer comprises the expression levels of the one or more genes or miRNAs and wherein the output layer comprises a classification of the expression level data of the input layer as indicating a canonical, an immune, or a stromal metastatic subtype. In some embodiments, step (b) comprises analyzing the expression levels of only genes listed in Table 1 and/or Table 2. In some embodiments, the appropriate therapy for a patient with a canonical-type metastasis comprises a DNA damaging chemotherapy, PARP inhibitor, angiogenesis inhibitor, or MYC inhibitor. In some embodiments, the appropriate therapy for a patient with an immune-type metastasis comprises cetuximab, immunotherapy, or a splicing inhibitor. In some embodiments, the appropriate therapy for a patient with a stromal-type metastasis comprises an angiogenesis inhibitor, KRAS inhibitor, or tumor stromal inhibitor, or excludes cetuximab.

Also disclosed is a method comprising evaluating expression levels of one or more genes listed in Table 1 and/or one or more miRNAs listed in Table 2 in a sample comprising tissue from a liver metastasis of a patient that has metastatic colorectal cancer to identify the patient as belonging to a first group of patients or a second group of patients, wherein said evaluating comprises using the expression levels as an input layer in a multi-layer neural network classification process and wherein: (a) the first group has one or more of the following characteristics: (i) a mean five-year overall survival expectation of at least 60%; (ii) a mean five-year overall survival expectation that is higher than that for patients outside of the first group; (iii) a likelihood of experiencing metastatic recurrence after hepatic resection that is lower than the likelihood for patients outside of the first group; (iv) a likelihood of being successfully treated without systemic cancer treatments that is higher than the likelihood for patients outside of the first group; (v) a likelihood of being successfully treated with immune checkpoint therapy that is higher than the likelihood for patients outside of the first group; (vi) a mean five-year disease-free survival expectation of greater than 30%; and (vii) a mean five-year disease-free survival expectation that is higher than that for patients outside of the first group; and (b) the second group has one or more of the following characteristics: (i) a mean five-year overall survival expectation of less than 60%; (ii) a mean five-year overall survival expectation that is lower than that for patients outside of the second group; (iii) a likelihood of experiencing metastatic recurrence after hepatic resection that is higher than for patients outside of the second group; (iv) a likelihood of being successfully treated without systemic cancer treatments that is lower than the likelihood for patients outside of the second group; (v) a likelihood of being successfully treated with immune checkpoint therapy that is lower than the likelihood for patients outside of the second group; (vi) a likelihood of being successfully treated with DNA damaging cancer therapy that is higher than the likelihood for patients outside of the second group; (vii) a mean five-year disease-free survival expectation of less than 30%; and (viii) a mean five-year disease-free survival expectation that is lower than that for patients outside of the second group. In some embodiments, the expression levels of the genes comprise expression levels of transcripts of at least 2, 3, 4, 5, 6, 7, 8, 9, 10, 11, 12, 13, 14, 15, 16, 17, 18, 19, 20, 21, 22, 23, or 24 of the genes listed in Table 1. In some embodiments, the expression levels of the miRNA species comprise expression levels of at least 2, 3, 4, 5, 6, or 7 of the miRNAs listed in Table 2. In some embodiments, the expression levels of the genes comprise all 24 of the genes listed in Table 1, and the expression levels of the miRNAs comprise all 7 of the miRNAs listed in Table 2. In some embodiments, only genes listed in Table 1 and only miRNAs listed in Table 2 are evaluated. In some embodiments, the patient is identified as belonging to the first group of patients if the neural network classification process indicates that the metastasis has a canonical or immune phenotype. In some embodiments, the patient is identified as belonging to the second group of patients if the neural network classification process indicates that the metastasis has a stromal phenotype. In some embodiments, the method further comprises administering an immune checkpoint therapy or cetuximab to a patient identified as belonging to the first group. In some embodiments, the method further comprises treating a patient identified as belonging to the first group with local treatment of liver metastases unaccompanied by systemic cancer treatment. In some embodiments, the method further comprises administering a DNA damaging cancer therapy to a patient identified as belonging to the second group of patients.

Also disclosed is a method of diagnosing and treating a patient having a metastasis from a primary colorectal cancer tumor, the method comprising: (a) measuring the expression of one or more genes listed in Table 1 or miRNAs listed in Table 2 in a sample from the metastasis; (b) identifying the metastasis as having a canonical, immune, or stromal phenotype based on the measured expression levels using a neural network classification system; and (d) administering to the patient an appropriate therapy based on the type of metastasis identified in step (c). In some embodiments, step (a) comprises measuring the expression of at least 2, 3, 4, 5, 6, 7, 8, 9, 10, 11, 12, 13, 14, 15, 16, 17, 18, 19, 20, 21, 22, 23, or 24 of the genes listed in Table 1 and/or at least 2, 3, 4, 5, 6, or 7 of the miRNAs listed in Table 2. In some embodiments, the appropriate therapy for a patient with a canonical-type metastasis comprises a DNA damaging chemotherapy, PARP inhibitor, angiogenesis inhibitor, or MYC inhibitor. In some embodiments, the appropriate therapy for a patient with an immune-type metastasis comprises cetuximab, immunotherapy, or a splicing inhibitor. In some embodiments, the appropriate therapy for a patient with a stromal-type metastasis comprises an angiogenesis inhibitor, KRAS inhibitor, or tumor stromal inhibitor, or excludes cetuximab.

Also disclosed is a method of treating a patient having metastatic colorectal cancer, the method comprising administering cetuximab to a patient who has been tested and found to have liver metastases of an immune molecular subtype. In some embodiments, the test comprises analyzing the expression levels of transcripts of at least 1, 2, 3, 4, 5, 6, 7, 8, 9, 10, 11, 12, 13, 14, 15, 16, 17, 18, 19, 20, 21, 22, 23, or 24 of the genes listed in Table 1 and at least 1, 2, 3, 4, 5, 6, or 7 of the miRNAs listed in Table 2. In some embodiments, the expression levels of the genes and miRNAs are analyzed using a neural network classification process. In some embodiments, the input into the neural network classification process consists of 1, 2, 3, 4, 5, 6, 7, 8, 9, 10, 11, 12, 13, 14, 15, 16, 17, 18, 19, 20, 21, 22, 23, or 24 of the genes listed in Table 1 and 1, 2, 3, 4, 5, 6, or 7 of the miRNAs listed in Table 2, or any combination of the genes listed in Table 1 and the miRNAs listed in Table 2. In some embodiments, the input into the neural network classification process consists of the 24 genes listed in Table 1 and the 7 miRNAs listed in Table 2.

A method of treating a patient having metastatic colorectal cancer, the method comprising administering a local cancer therapy unaccompanied by systemic cancer therapy to a patient who has been tested and found to have liver metastases of a canonical or immune molecular subtype, wherein the test comprises analyzing the expression levels of transcripts of one or more genes listed in Table 1 and one or more miRNAs listed in Table 2 using a neural network classification process. In some embodiments, the input into the neural network classification process includes only genes listed in Table 1 and only miRNAs listed in Table 2. In some embodiments, the input into the neural network classification process consists of the 24 genes listed in Table 1 and the 7 miRNAs listed in Table 2.

Also disclosed is a method of providing a prognosis for a patient having metastatic colorectal cancer, the method comprising: (a) evaluating the expression of one or more genes listed in Table 1 and/or one or more miRNAs listed in Table 2 in a tissue sample from a metastasis taken from the patient to identify the metastasis as a canonical, immune, or stromal-type metastasis; (b) determining the clinical risk score of the patient; (c) determining the ten-year survival expectation of the patient as follows: (i) identifying the patient as having a ten-year overall survival expectation of greater than 90% if the metastasis is canonical or immune and the clinical risk score is 0 or 1; (ii) identifying the patient as having a ten-year survival expectation of between 40 and 50% if the metastasis is immune-type and the clinical risk score is 2 or greater or if the metastasis is type stromal and the clinical risk score is 0 or 1; and (iii) identifying the patient as having a ten-year survival expectation of less than 24% if the metastasis is canonical or stromal and the clinical risk score is 2 or greater. In some embodiments, the genes and/or miRNAs comprise at least, at most, or exactly 1, 2, 3, 4, 5, 6, 7, 8, 9, 10, 11, 12, 13, 14, 15, 16, 17, 18, 19, 20, 21, 22, 23, or 24 of the genes listed in Table 1, or any range derivable therein, and at least, at most, or exactly 1, 2, 3, 4, 5, 6, or 7 of the miRNAs listed in Table 2, or any range derivable therein.

In any of the embodiments described herein, gene expression analysis can be performed using a classifier that was trained using a neural network process having as inputs at least, at most, or exactly 1, 2, 3, 4, 5, 6, 7, 8, 9, 10, 11, 12, 13, 14, 15, 16, 17, 18, 19, 20, 21, 22, 23, or 24 of the genes listed in Table 1, or any range derivable therein, and at least, at most, or exactly 1, 2, 3, 4, 5, 6, or 7 of the miRNAs listed in Table 2, or any range derivable therein. In some embodiments, the trained classifier assigns a probability that a given set of expression levels represents an expression signature of a canonical, immune, or stromal molecular subtype. In some embodiments, the expression signatures were previously determined by a neural network classification process. In some embodiments, the trained classifier compares input expression levels of the genes and miRNAs to reference expression levels of the genes and miRNAs, wherein the reference expression levels were determined using a neural network classification process. In some embodiments, the trained classifier compares input expression levels of the genes and miRNAs to reference expression signatures for canonical, immune, and/or stromal metastatic subtypes.

Also disclosed is a method of diagnosing a patient having a liver metastasis from a primary colorectal cancer tumor, the method comprising inputting the expression levels in the metastasis of one or more of the genes listed on Table 1 and one or more of the miRNAs listed in Table 2 into a classifier that has been trained to recognize an expression signature of a canonical, immune, and/or stromal metastatic molecular subtype. In some embodiments, the classifier is configured to recognize an expression signature of a canonical, immune, and/or stromal metastatic molecular subtype. In some embodiments, the classifier is configured to assign a probability that the input expression levels are from a canonical, immune, and/or stromal metastatic molecular subtype. In some embodiments, the classifier has been trained using a neural network machine learning process. In some embodiments, the expression levels of all 24 of the genes listed on Table 1 and all 7 of the miRNAs listed on Table 1 are inputted into the classifier. In some embodiments, no other expression levels are input into the classifier.

Also disclosed is a method of treating metastatic colorectal cancer in a patient, the method comprising administering to the patient a local cancer therapy unaccompanied by systemic cancer therapy, wherein the patient has been identified as having metastases of a canonical or immune subtype by a classifier that analyzed expression levels in a metastasis tissue sample from the patient of one or more of the genes listed in Table 1 and one or more of the miRNAs listed in Table 2, wherein the classifier was configured to recognize an expression signature of a canonical or immune subtype based on the expression levels. In some embodiments, the classifier was configured to assign a probability that the expression levels represent an expression signature of a canonical, immune, or stromal molecular subtype. In some embodiments, the only metastasis expression levels analyzed by the classifier are the 24 genes listed in Table 1 and the 7 miRNAs listed in Table 2. In some embodiments, the classifier has been trained using a neural network machine learning process.

In any of the embodiments described herein, the patient may have already been diagnosed with cancer or already had tumor resection before any of the steps of methods described herein are performed.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Any step or aspect of an embodiment described herein may be implemented in the context of any other embodiment described herein.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that embodiments described herein in the context of the term “comprising” may also be implemented in the context of the term “consisting of” or “consisting essentially of.”

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary, Detailed Description, Claims, and Brief Description of the Drawings.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 illustrates a neural network classification process.

FIGS. 2A-2B show a comparison of the molecular subtypes (FIG. 2A) and clinical risk classifications (FIG. 2B) in the UK study and 2018 study cohorts. The data for the UK study cohort are labeled “UK,” and the data for the 2018 study cohort are labeled “UCMC.”

FIG. 3 shows Kaplan-Meier curves for disease-free survival (left panel) and overall survival (right panel) in the UK cohort for patients with metastases having low and intermediate risk classification according to the integrated risk group classification, as compared to patients with metastases having a high risk classification.

FIGS. 4A-4B: Imbalance of molecular subtypes by treatment arm. FIG. 4A: comparison of molecular subtype classification in the two treatment arms of the UK cohort (+/−cetuximab). FIG. 4B: Comparison of KRAS signaling phenotype in different molecular subtypes in the cetuximab arm of the UK cohort.

FIG. 5 shows Kaplan-Meier curves for disease-free survival for the indicated molecular subtypes (canonical, immune, or stromal) and treatment arms (cetuximab +or −) in the UK cohort.

FIG. 6 shows a diagram representing training and application of neural network classifier to predict molecular subtypes.

FIG. 7A shows a single-sample gene set enrichment analysis across molecular subtypes in the validation cohort; FIG. 7B shows immune deconvolution across molecular subtypes in the validation cohort.

FIGS. 8A-8D show survival outcomes in validation cohort; (FIG. 8A) PFS by molecular subtype (FIG. 8B) OS by molecular subtype (FIG. 8C) PFS by integrated risk group (FIG. 8D) OS by integrated risk group.

FIG. 9 shows optimization of model performance (measured by the F score) as features are eliminated using recursive feature elimination.

FIG. 10 shows a histogram representing the robustness and internal consistency of the molecular subtype classifier for liver metastases in the validation cohort. Of 100 neural network models applied for each specimen in the classifier, the distribution of model concordance for predicting molecular subtypes is visualized. P value corresponds to single sample T-test (N=147) with Ha: mean percentage of concordant models across participants >33.3% (i.e. better than a truly random classifier).

FIGS. 11A-11B show distribution of molecular subtypes and integrated clinical-molecular risk groups in the discovery and validation cohorts.

FIG. 12 shows distribution of clinical and pathologic features across molecular subtypes in the validation cohort.

FIG. 13A shows PFS and OS for overall discovery and validation cohorts; FIG. 13B shows OS for canonical, immune, and stromal subtypes; FIG. 13C shows OS for low-risk, intermediate-risk, high-risk integrated risk groups.

FIGS. 14A-14B show survival outcomes in validation cohort by predicted molecular subtype of primary tumor; (FIG. 14A) PFS; (FIG. 14B) OS.

FIGS. 15A-15D show survival outcomes in the validation cohort based on consensus molecular subtypes of either the primary tumor or liver metastasis; (FIG. 15A) PFS by primary tumor CMS; (FIG. 15B) OS by primary tumor CMS; (FIG. 15C) PFS by liver metastasis CMS; (FIG. 15D) OS by liver metastasis CMS.

### DETAILED DESCRIPTION

Here, utilizing a prospective clinical cohort of CRC patients who underwent resection of liver metastases, the inventors have identified and validated expression signatures of molecular subtypes of colorectal cancer liver metastases (CRCLM) using fewer expression inputs than was previously possible. The inventors' findings validate a molecular basis for oligometastasis that is predictive of clinical outcome and complementary to established clinical risk factors associated with long-term survival following hepatic resection. Aspects of the current disclosure have important clinical implications in the selection of local therapy for those patients with potentially curable oligometastatic disease from those whose few metastases are a part of a large cascade of widespread disease. These concepts may be applicable to many histological types of cancer. Methods disclosed herein involve determining expression levels of genes and miRNAs in liver metastases to identify the molecular subtype of the metastasis. The subtype classification can be used to provide a prognosis and to guide treatment decisions. These and other aspects of the disclosed methods will be described in greater detail below.

### A. Gene and miRNA Expression Levels

Methods disclosed herein include measuring expression of genes and/or miRNAs. Measurement of expression can be done by a number of processes known in the art. The process of measuring expression may begin by extracting RNA from a metastasis tissue sample. Extracted mRNA and/or miRNA can be detected by hybridization (for example by means of Northern blot analysis or DNA or RNA arrays (microarrays) after converting mRNA into labeled cDNA) and/or amplification by means of an enzymatic chain reaction. Quantitative or semi-quantitative enzymatic amplification methods such as polymerase chain reaction (PCR) or quantitative real-time RT-PCR or semi-quantitative RT-PCR techniques can be used. Primer pairs may be designed for the purpose of superimposing an intron to distinguish cDNA amplification from the contamination from genomic DNA (gDNA). Additional primers or probes, which are preferably labeled, for example with fluorescence, which hybridize specifically in regions located between two exons, are optionally designed for the purpose of distinguishing cDNA amplification from the contamination from gDNA. If desired, said primers can be designed such that approximately the nucleotides comprised from the 5′ end to half the total length of the primer hybridize with one of the exons of interest, and approximately the nucleotides comprised from the 3′ end to half the total length of said primer hybridize with the other exon of interest. Suitable primers can be readily designed by a person skilled in the art. Other amplification methods include ligase chain reaction (LCR), transcription-mediated amplification (TMA), strand displacement amplification (SDA) and nucleic acid sequence based amplification (NASBA). Expression levels of mRNAs and/or miRNAs may also be measured by RNA sequencing methods known in the art.

To normalize the expression values of one gene among different samples, comparing the mRNA level of the gene of interest in the samples from the subject object of study with a control RNA level is possible. As it is used herein, a “control RNA” is an RNA of a gene for which the expression level does not differ among different metastatic subtypes, for example a gene that is constitutively expressed in all types of cells. A control RNA is preferably an mRNA derived from a housekeeping gene encoding a protein that is constitutively expressed and carrying out essential cell functions.

Methods disclosed herein may include comparing a measured expression level to a reference expression level. The term “reference expression level” refers to a value used as a reference for the values/data obtained from samples obtained from patients. The reference level can be an absolute value, a relative value, a value which has an upper and/or lower limit, a series of values, an average value, a median, a mean value, or a value expressed by reference to a control or reference value. A reference level can be based on the value obtained from an individual sample, such as, for example, a value obtained from a sample from the subject object of study but obtained at a previous point in time. The reference level can be based on a high number of samples, such as the levels obtained in a cohort of subjects having a particular characteristic. The reference level may be defined as the mean level of the patients in the cohort. For example, the reference expression level for a gene or miRNA can be based on the mean expression level of the gene or miRNA obtained from a number of patients who have immune subtype metastases. A reference level can be based on the expression levels of the markers to be compared obtained from samples from subjects who do not have a disease state or a particular phenotype. The person skilled in the art will see that the particular reference expression level can vary depending on the specific method to be performed.

Some embodiments include determining that a measured expression level is higher than, lower than, increased relative to, decreased relative to, equal to, or within a predetermined amount of a reference expression level. In some embodiments, a higher, lower, increased, or decreased expression level is at least 1, 1.5, 2, 2.5, 3, 3.5, 4, 4.5, 5, 5.5, 6, 7, 8, 9, 10, 12, 14, 16, 18, 20, 50, 100, 150, 200, 250, 500, or 1000 fold (or any derivable range therein) or at least 10, 20, 30, 40, 50, 60, 70, 80, 90, 100, 200, 300, 400, 500, 600, 700, 800, or 900% different than the reference level, or any derivable range therein. These values may represent a predetermined threshold level, and some embodiments include determining that the measured expression level is higher by a predetermined amount or lower by a predetermined amount than a reference level. In some embodiments, a level of expression may be qualified as “low” or “high,” which indicates the patient expresses a certain gene or miRNA at a level relative to a reference level or a level with a range of reference levels that are determined from multiple samples meeting particular criteria. The level or range of levels in multiple control samples is an example of this. In some embodiments, that certain level or a predetermined threshold value is at, below, or above 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percentile, or any range derivable therein. Moreover, a threshold level may be derived from a cohort of individuals meeting a particular criteria. The number in the cohort may be, be at least, or be at most 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 700, 800, 900, 1000, 1100, 1200, 1300, 1400, 1500, 1600, 1700, 1800, 1900, 2000 or more (or any range derivable therein). A measured expression level can be considered equal to a reference expression level if it is within a certain amount of the reference expression level, and such amount may be an amount that is predetermined. This can be the case, for example, when a classifier is used to identify the molecular subtype of a metastasis. The predetermined amount may be within 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19, 20, 21, 22, 23, 24, 25, 30, 35, 40, 45, or 50% of the reference level, or any range derivable therein.

For any comparison of gene or miRNA expression levels to a mean expression levels or a reference expression levels, the comparison is to be made on a gene-by-gene and miRNA-by-miRNA basis. For example, if the expression levels of gene A, gene B, and miRNA X in a patient's metastasis are measured, a comparison to mean expression levels in metastases of a cohort of patients would involve: comparing the expression level of gene A in the patient's metastasis with the mean expression level of gene A in metastases of the cohort of patients, comparing the expression level of gene B in the patient's metastasis with the mean expression level of gene B in metastases of the cohort of patients, and comparing the expression level of miRNA X in the patient's metastasis with the mean expression level of miRNA X in metastases of the cohort of patients. Comparisons that involve determining whether the expression level measured in a patient's metastasis is within a predetermined amount of a mean expression level or reference expression level are similarly done on a gene-by-gene and miRNA-by-miRNA basis, as applicable.

### B. Identifying Molecular Subtypes of Metastases

Methods disclosed herein can be used to identify different molecular subtypes of metastatic cancer that correlate with different clinical outcomes and different sensitivities to particular treatment regimens. The subtypes can be identified using a multi-layer neural network classification technique.

A neural network is a machine learning computing system that consist of a number of simple but highly interconnected elements or nodes, called ‘neurons’, which are organized in layers which process information using dynamic state responses to external inputs. Neural network systems are useful in finding expression signatures that are too complex to be manually derived and taught to a machine. A neural network can be constructed for a selected set of expression levels. In multilayer neural networks, there are input units (input layer), hidden units (hidden layer), and output units (output layer). There is, furthermore, a single bias unit that is connected to each unit other than the input units. Neural networks are described in Duda et al., 2001, Pattern Classification, Second Edition, John Wiley & Sons, Inc., New York; and Hastie et al., 2001, The Elements of Statistical Learning, Springer-Verlag, New York.

### C. Cancer Treatment

Methods disclosed herein may include administering a cancer therapy or determining a course of cancer treatment based on an identified metastatic subtype. Some embodiments include administering a local cancer treatment or determining that a local cancer treatment is appropriate. Local cancer treatments include those that target cancer tissue using a technique directed to a specific organ or limited area of the body. Local cancer treatments include surgery (i.e., resection), radiation therapy, cryotherapy, laser therapy, topical therapy, high intensity focused ultrasound, and photodynamic therapy. The local treatments may include stereotactic body radiotherapy (SBRT), stereotactic ablative body radiotherapy (SABR), stereotactic radiosurgery (SRS), radiofrequency ablation (RFA), percutaneous cryoablation therapy (PCT), and photodynamic therapy (PDT). The local therapies may be directed at the primary tumor and/or at one or more metastases.

Systemic cancer therapies are those that are distributed widely within the body, such as a variety of drug treatments, which may be delivered orally or intravenously. Examples of systemic therapies include chemotherapy, hormone therapy, immunotherapy, and targeted therapy (i.e., drugs that are distributed widely within the body, but have targeted effects on cancer cells). More specifically, chemotherapy includes administering drugs such as cyclophosphamide, paclitaxel, epirubicin, methotrexate, gemcitabine, albumin-bound paclitaxel, carboplatin, etoposide, doxorubicin, capecitabine, fluorouracil, vinorelbine, docetaxel, liposomal doxorubicin, eribulin, or irinotecan, including combinations thereof. Immunotherapy includes monoclonal antibodies, such as alemtuzumab, trastuzumab, ibritumomab tiuxetan, brentuximab vedotin, ado-trastuzumab emtansine, denileukin diftitox, and blinatumomab; immune checkpoint inhibitors, such as pembrolizumab, nivolumab, atezolizumab, avelumab, durvalumab, and ipilimumab; and cancer vaccines such as sipuleucel-T.

Identifying the molecular subtype of metastatic colorectal cancer can be used to determine an appropriate treatment regimen. In some embodiments, the appropriate treatment for canonical subtype metastases include EGFR inhibitors (e.g., cetuximab, panitumumab); PARP inhibitors; PI3K inhibitors; NOTCH inhibitors; angiogensis inhibitors; DNA damaging agents such as cisplatin, oxaliplatin, carboplatin, cyclophosphamide, chlorambucil, or temozolomide; STING agonists; innate immune agonists; RNA vaccines; MYC inhibitors; or combinations thereof. In some embodiments, the appropriate treatment for immune subtype metastases include EGFR inhibitors (e.g., cetuximab, panitumumab), PD-1/PD-L1 immunotherapies, other immunotherapies, beta-secretase inhibitors, lipid-lowering agents, splicing inhibitors, and combinations thereof. In some embodiments, the appropriate treatment for stromal subtype metastases include PDGF/PDGFR inhibitors, KRAS inhibitors, tumor stromal inhibitors, VEGF/VEGFR inhibitors, angiogenesis inhibitors, JAK1/JAK2 inhibitors, COX2 inhibitors, HDAC inhibitors, DNA demethylating agents, other epigenetic modifiers, and combinations thereof. In some embodiments, the appropriate treatment for stromal subtype metastases excludes cetuximab and/or panitumumab.

In some embodiments, methods herein include administering an EGFR inhibitor (e.g., cetuximab, a monoclonal antibody that binds epidermal growth factor receptor (EGFR)), to patients depending on the molecular subtype of their metastases. In some embodiments, the EGFR inhibitor (e.g., cetuximab) is administered to patients who have been tested and determined to have immune molecular subtype metastases. In some embodiments, the EGFR inhibitor (e.g., cetuximab) is administered weekly or every other week. In some embodiments, an initial dose of 400 mg/m² is administered, followed by weekly doses of 250 mg/m². In some embodiments, the initial dose is at least about, at most about, or about 100, 150, 200, 250, 300, 350, 400, 450, or 500 mg/m², or is between any two of these values. In some embodiments, the subsequent weekly doses are at least about, at most about, or about 50, 100, 150, 200, 250, 300, 350, or 400 mg/m², or are between any two of these values. The doses may be infused over the course of 1 to 2 hours at an infusion rate of no more than 10 mg/min. In some embodiments, the patient is tested and determined to have a KRAS wild type genotype.

In some embodiments, panitumumab, another EGFR receptor-binding monoclonal antibody, is administered to a patient who has immune molecular subtype metastases. In some embodiments, the dosage administered is 6 mg/kg every other week. In some embodiments, the dosage is at least about, at most about, or about 1, 2, 3, 4, 5, 6, 7, 8, 9, or 10 mg/kg every other week, or is between any two of these values.

Methods disclosed herein can also include making treatment decisions based on an integrated risk group classification of a patient. This classification combines the molecular subtyping of the metastasis with a clinical risk score of the patient and divides patients into low risk, intermediate risk, and high risk groups based on their respective five-year probabilities of disease-free survival or overall survival. A patient's integrated risk group indicates the likelihood of benefit from local metastasis-directed therapies such as surgical resection, stereotactic body radiotherapy (SBRT), stereotactic ablative body radiotherapy (SABR), stereotactic radiosurgery (SRS), radiofrequency ablation (RFA), percutaneous cryoablation therapy (PCT), and photodynamic therapy (PDT): low-risk patients have the highest likelihood of benefit from these therapies, high-risk patients have the lowest likelihood of benefit from these therapies, and intermediate-risk patients have an intermediate likelihood of benefit from these therapies.

Conventionally, it has been thought that metastatic cancer always requires a systemic therapy. However, determination of the molecular subtypes of metastatic cancer as described herein can be used to indicate metastatic cancers, such as those with canonical or immune subtype metastases, are likely to respond favorably to local therapies and may not need an additional systemic therapy. Conversely, some metastatic cancers, such as those with stromal subtype metastases, are not likely to respond to local therapy alone, or at all, and should therefore be treated with appropriate systemic therapies.

### D. Examples

### Example 1. Clinical Characteristics and Patient Outcomes

The inventors previously identified three molecular subtypes of colorectal liver metastases (CRCLM) designated as canonical (SNF1), immune (SNF2), and stromal (SNF3) subtypes. See Pitroda et al., “Integrated molecular subtyping defines a curable oligometastatic state in colorectal liver metastasis,” Nature Communications 9:1793 (2018) (hereinafter, “Pitroda 2018 Publication”); WO2019/204576. The purpose of the current study was to develop an efficient classification process using fewer expression level inputs and to validate the existence of and prognostic differences between these three molecular subtypes in an independent clinical cohort.

Few retrospective cohorts of CRCLM are available, and those that are available are limited by small sample sizes and non-randomized selection of patients for analysis. Herein is presented a validation of the molecular subtypes in a randomized clinical trial from the UK using a unique neural network-based classifier. In this trial, patients received standard of care pre-operative/post-operative chemotherapy, including neoadjuvant plus adjuvant chemotherapy and complete resection of all CRCLM. Primary tumors were also treated with curative intent. The study included 257 KRAS wild-type (codons 12, 13, & 61) colorectal cancer patients, and 80% of the patients had 1 to 3 liver metastases. The randomization was to +/− cetuximab in the pre-operative and post-operative settings. Ultimately, the trial was negative for the primary endpoint. Microarray profiling of 147 CRCLM specimens for gene expression were performed using the Affymetrix Xcel microarray platform. Mutational and CNV analyses were also performed.

The inventors' prior studies found that mRNA data alone or miRNA data alone were insufficient to classify patients into the three molecular subtypes of CRCLM. By contrast, integration of both mRNA and miRNA data accurately classified the molecular subtypes of CRCLM. In the present study, the inventors aimed to minimize the number of input mRNA and miRNA features while maintaining a high accuracy for classification into the three molecular subtypes. The inventors first overlapped the mRNA and miRNA features that were present in the Pitroda 2018 Publication with the data from the UK randomized trial Xcel platform. This provided the full set of potential input mRNA and miRNA features. The inventors utilized a neural network classifier (a machine learning algorithm) to derive a classifier in the cohort from the Pitroda 2018 Publication that could then be validated in the UK validation cohort. In this context, 2018 study cohort was split into a training and testing set (60% and 40% of samples respectively) from which a signature was discovered and iteratively optimized. The model was first derived by training the neural network containing a hidden layer of 25 neurons and using as the input standardized z-scores of 400 mRNA and 41 miRNA expression values for each patient in the 2018 study cohort. The 400 mRNAs were selected from approximately 20,000 mRNAs on the basis of having the highest principal components (PC1 and PC2) using a principal components analysis. The 41 miRNAs were selected as being present in both platforms used in the 2018 study and the UK study. At this initial stage the average model accuracy using 400 mRNAs and 41 miRNAs as input features was 83% in the 2018 cohort testing set. In order to improve the model prediction, a recursive feature elimination was performed where input features that did not contribute significantly to the model accuracy were successively eliminated. The final model contained only 24 mRNAs (listed in Table 1 below) and 7 miRNAs (listed in Table 2 below), for a total of only 31 features.

TABLE 1
mRNAs included in final model

| | Gene |
|---|---|
| ENSEMBL ID | Symbol |
| ENSG00000138138 | ATAD1 |
| ENSG00000115816 | CEBPZ |
| ENSG00000164323 | CFAP97 |
| ENSG00000119878 | CRIPT |
| ENSG00000162733 | DDR2 |
| ENSG00000105722 | ERF |
| ENSG00000078098 | FAP |
| ENSG00000114933 | INO80D |
| ENSG00000150995 | ITPR1 |
| ENSG00000160593 | JAML |
| ENSG00000169744 | LDB2 |
| ENSG00000171488 | LRRC8C |
| ENSG00000187098 | MITF |
| ENSG00000121879 | PIK3CA |
| ENSG00000118762 | PKD2 |
| ENSG00000046889 | PREX2 |
| ENSG00000146282 | RARS2 |
| ENSG00000134597 | RBMX |
| ENSG00000084093 | REST |
| ENSG00000080345 | RIF1 |
| ENSG00000163785 | RYK |
| ENSG00000110719 | TCIRG1 |
| ENSG00000184281 | TSSC4 |
| ENSG00000115464 | USP34 |

TABLE 2
miRNAs included in final model

| | miRNA |
|---|---|
| ENSEMBL ID | Symbol |
| ENSG00000284190 | MIR21 |
| ENSG00000207962 | MIR30C1 |
| ENSG00000265841 | MIR548X |
| ENSG00000283394 | MIR7515 |
| ENSG00000211591 | MIR762 |
| ENSG00000284425 | MIR8072 |
| ENSG00000284586 | MIR92B |

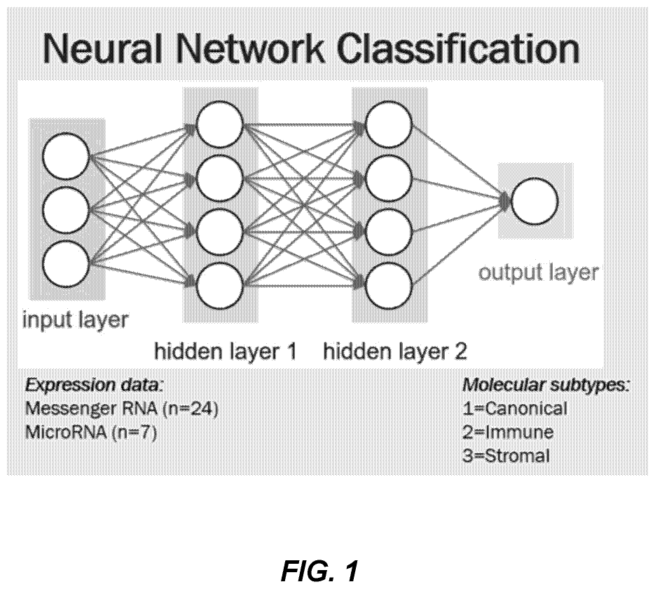

The average model accuracy using the set of 31 features was 96% in the 2018 study testing cohort. Using randomly selected sets of 60% of the input data (UK cohort), 100 independent neural network models were generated to predict the three molecular subtypes. Each model provides an output for the probability that a given sample corresponds to the canonical, immune, and stromal subtypes. Using the set of 100 trained neural network models, a subtype classification was performed on the UK cohort where the final subtype for each sample corresponds to the most frequent subtype chosen by the 100 models. FIG. 1 shows a schematic of a neural network classification model. The input layer comprises input data such as mRNA or miRNA expression data. The classification model can have multiple hidden layers, each with a number of nodes, or neurons. The output layer provides probabilities that the input data fits into one or more classes, such as one or more of the three molecular subtypes of CRCLM.

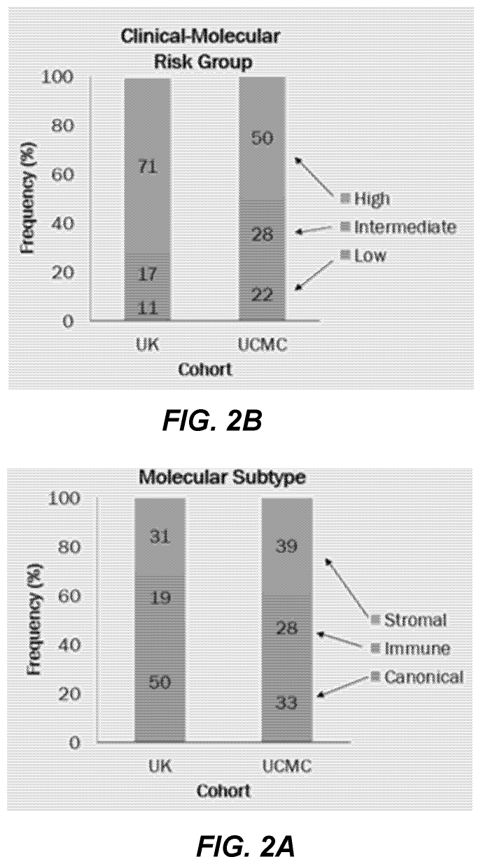

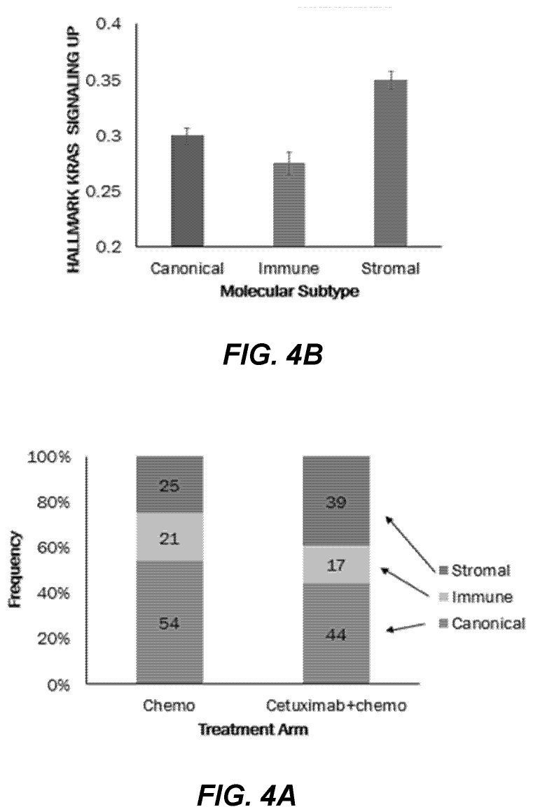

FIGS. 2A and 2B show a comparison of the molecular subtypes of the CRCLM samples in the UK study cohort (labeled “UK” in FIGS. 2A and 2B) and the Pitroda 2018 Publication study cohort (labeled “UCMC” in FIGS. 2A and 2B). The distribution of the CRCLM molecular subtypes is different across the UK and Pitroda 2018 Publication cohorts with greater frequencies of the adverse subtypes (canonical and stromal) in the UK cohort (FIG. 2A). Moreover, the inventors previously proposed an integrated risk classification based on molecular subtypes and clinical risk scores (Pitroda 2018 Publication, FIG. 4). The distribution of the integrated risk groups in the UK cohort was examined, and significantly fewer low risk patients and much higher frequency of high risk patients (i.e. patients who are likely to have poor clinical outcomes after treatment) were found (FIG. 2B).

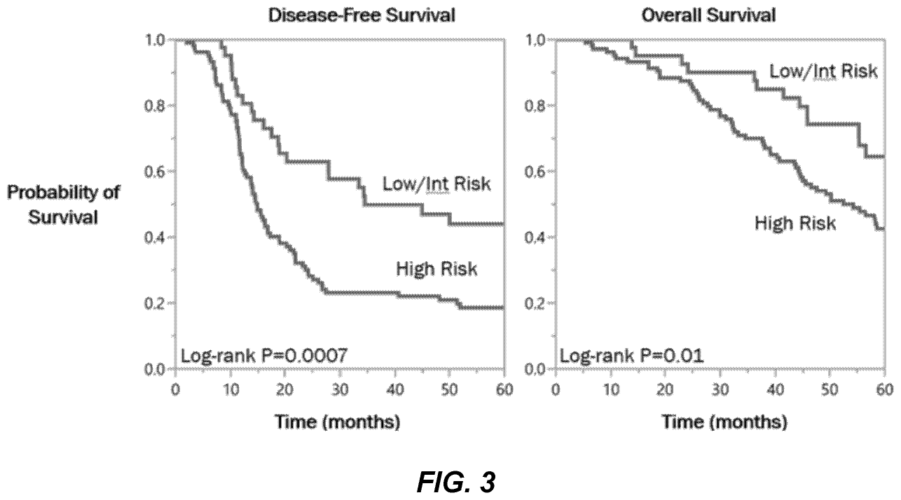

Importantly, patients in the UK cohort had significantly different disease free and overall survival based on the integrated risk group classification (FIG. 2B). FIG. 3 shows that patients in the low+intermediate risk group using the integrated risk group classification have nearly 25% (absolute) improvements in disease free and overall survivals as compared to high risk patients. This is a direct validation of the existence and prognostic impact of the molecular subtypes identified herein in a prospective clinical cohort.

Given that the UK trial was negative for its primary endpoint of a disease free survival benefit with the addition of cetuximab to standard chemotherapy, the inventors tested whether the CRCLM molecular subtypes could provide an explanation for their clinical outcomes. A statistical imbalance between the standard of care chemotherapy arm and the chemotherapy+cetuximab arm was found, with more stromal (adverse subtype) patients in the cetuximab arm (FIG. 4A). Moreover, the tumors exhibiting the stromal phenotype had increased KRAS signaling activation (FIG. 4B), which is a known resistance mechanism to cetuximab.

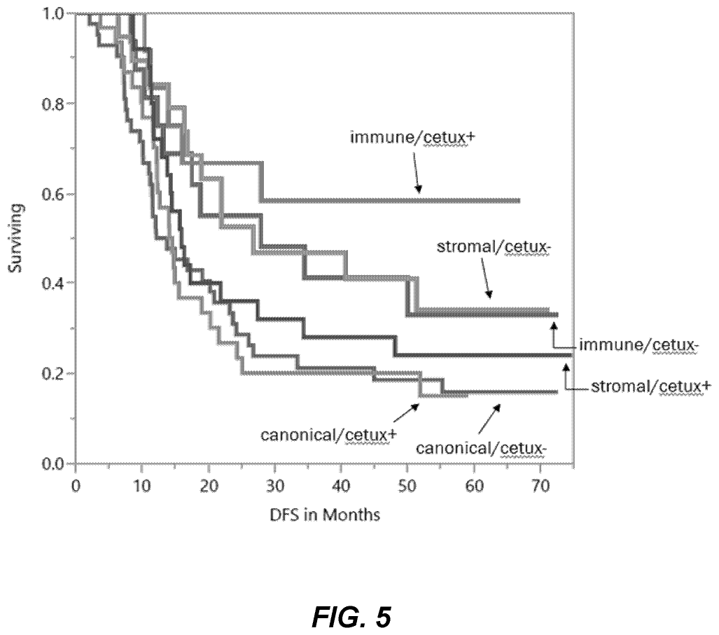

The inventors determined the disease-free survival Kaplan-Meier curves for the three molecular subtypes in the two treatment arms in the UK study (cetuximab +or −) (see FIG. 5). Patients with CRCLM tumors of the canonical molecular subtype showed no difference in disease free survival with or without cetuximab. Patients with CRCLM tumors of the immune subtype had an improvement in disease free survival with cetuximab, indicating that cetuximab would be clinically useful for this subset of patients. By contrast, patients with CRCLM tumors of the stromal subtype had a detriment in disease-free survival with cetuximab. The patients treated with cetuximab were more likely to develop widespread recurrences after their initial treatment, which may be due to cetuximab treatment selecting pre-existing tumor clones or causing the emergence of drug resistant tumor clones due to elevated KRAS signaling in these tumors, leading to increased distant metastasis and death in patients with the stromal CRCLM subtype.

### Example 2. Neural Network Classifier

The inventors developed a neural network classifier based on expression of the 31 features identified in Table 1 and Table 2. In summary, the expression feature inputs (X) from a sample plus a column of 1's get matrix multiplied by a transposed Theta1 (see Table 3 below), and this gives the matrix h1. This matrix is then fed into a sigmoid function and the output plus a column of 1's gets multiplied by the transposed Theta2 (see Table 3 below) and fed to a sigmoid. The final result is a column vector of three probabilities giving the probability of subtype 1 (canonical), 2 (immune), or 3 (stromal). The final subtype classification output is determined by assigning the sample to the class corresponding to the highest probability.

The Theta1 matrices have an additional column that corresponds to the bias term. This is a constant feature input that is always 1, it is analogous to a constant term for a linear or logistic regression. The Theta 2 matrices also have 36 columns corresponding to the 35 neurons used in the hidden layer plus an additional bias term of 1. The inputs to the output layer is the output of the hidden layer plus the constant bias term. That input is fed into 3 output neurons that give the probability of the sample being of class 1 (canonical), 2 (immune), or 3 (stromal). Below is the matlab code used to calculate the prediction:

function p = predict(Thetal, Theta2, X)
%PREDICT Predict the label of an input given a trained neural network
% p = PREDICT(Theta1, Theta2, X) outputs the predicted label of X given the
% trained weights of a neural network (Thetal, Theta2)
% Useful values
m = size(X, 1);
num labels = size(Theta2, 1);
% You need to return the following variables correctly
p = zeros(size(X, 1), 1);
h1 = sigmoid([ones(m, 1) X] * Theta1′);
h2= sigmoid([ones(m, 1) h1] * Theta2′);
[dummy, p] = max(h2, [ ], 2);
%
===============================================================
==========
end

TABLE 3
Theta1 and Theta2 for Neural Network Classifier

| Column ID | | | Column ID | |
|---|---|---|---|---|
| (Theta 1) | Feature | Gene Symbol | (Theta 2) | Feature |
| A | Bias term | | A | Bias term |
| B | ENSG00000160593.16 | JAML | B | Node 1 |
| C | ENSG00000046889.17 | PREX2 | C | Node 2 |
| D | ENSG00000078098.12 | FAP | D | Node 3 |
| E | ENSG00000187098.13 | MITF | E | Node 4 |
| F | ENSG00000169744.11 | LDB2 | F | Node 5 |
| G | ENSG00000171488.13 | LRRC8C | G | Node 6 |
| H | ENSG00000162733.15 | DDR2 | H | Node 7 |
| I | ENSG00000184281.13 | TSSC4 | I | Node 8 |
| J | ENSG00000119878.5 | CRIPT | J | Node 9 |
| K | ENSG00000110719.8 | TCIRG1 | K | Node 10 |
| L | ENSG00000118762.6 | PKD2 | L | Node 11 |
| M | ENSG00000150995.16 | ITPR1 | M | Node 12 |
| N | ENSG00000105722.8 | ERF | N | Node 13 |
| O | ENSG00000164323.11 | CFAP97 | O | Node 14 |
| P | ENSG00000146282.16 | RARS2 | P | Node 15 |
| Q | ENSG00000121879.3 | PIK3CA | Q | Node 16 |
| R | ENSG00000138138.12 | ATAD1 | R | Node 17 |
| S | ENSG00000115816.12 | CEBPZ | S | Node 18 |
| T | ENSG00000163785.11 | RYK | T | Node 19 |
| U | ENSG00000084093.14 | REST | U | Node 20 |
| V | ENSG00000080345.16 | RIF1 | V | Node 21 |
| W | ENSG00000115464.13 | USP34 | W | Node 22 |
| X | ENSG00000114933.14 | INO80D | X | Node 23 |
| Y | ENSG00000147274.13 | RBMX | Y | Node 24 |
| Z | MIR548X | MIR548X | Z | Node 25 |
| AA | MIR21 | MIR21 | AA | Node 26 |
| AB | MIR8072 | MIR8072 | AB | Node 27 |
| AC | MIR762 | MIR762 | AC | Node 28 |
| AD | MIR92B.1 | MIR92B.1 | AD | Node 29 |
| AE | MIR7515 | MIR7515 | AE | Node 30 |
| AF | MIR30C.1 | MIR30C.1 | AF | Node 31 |
| | | | AG | Node 32 |
| | | | AH | Node 33 |
| | | | AI | Node 34 |
| | | | AJ | Node 35 |

### Example 3. Validation of an Integrated Clinical-Molecular Classification of Colorectal Liver Metastases: A Biomarker Analysis of the Randomized Phase III New EPOC Trial

These studies present validation of a 31-gene expression signature that accurately predicts the colorectal liver metastasis molecular subtypes as a secondary analysis of the large multicenter, randomized, controlled phase III New EPOC trial.^{9,10} Importantly, it was confirmed that integrated clinical-molecular risk groups are highly prognostic for survival. Taken together, these findings demonstrate a potential strategy to personalize treatment approaches for patients with limited liver metastases from colorectal cancer and confirm that a low-risk integrated subgroup achieves excellent overall survival after surgical resection.

### Results

### Patient Characteristics

The discovery cohort was comprised of 93 patients who underwent predominantly peri-operative 5-fluorouracil and platinum-based chemotherapy and hepatic resection, while the validation cohort was comprised of 147 patients randomized in the phase III New EPOC trial. Patient characteristics are summarized in Table 10. Overall, both cohorts were representative of patients who underwent hepatic resection for limited liver metastases from colorectal adenocarcinoma in the setting of peri-operative chemotherapy. However, patients in the validation cohort exhibited greater adverse risk factors for recurrence and death, such as increased age, synchronous presentation of liver metastases, and high Clinical Risk Scores.

### Training a Molecular Subtype Classifier in Discovery Cohort

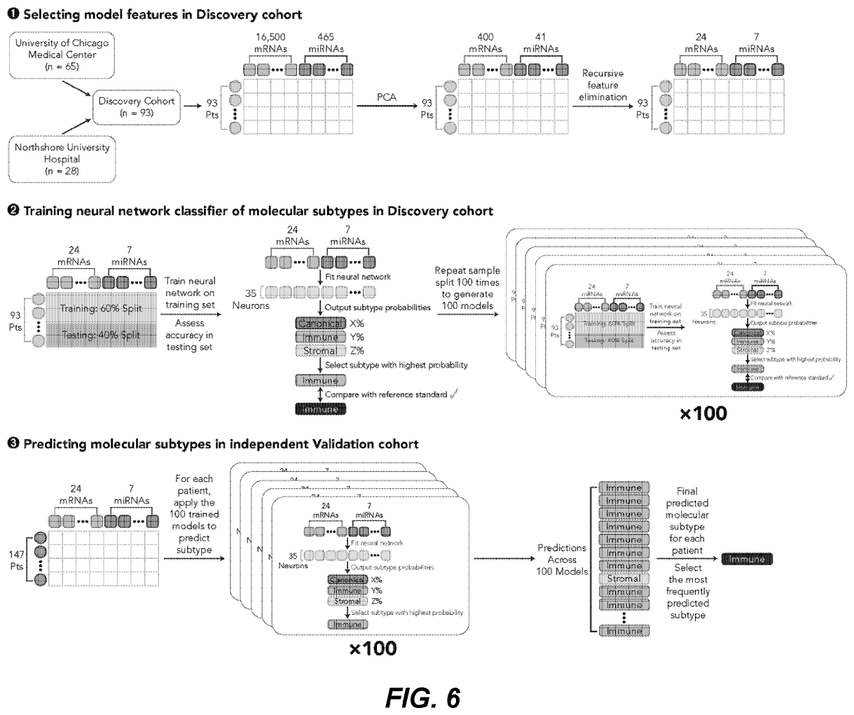

Expression data in the discovery cohort was based on whole transcriptome RNA sequencing and miRNA profiling, comprising 17,162 mRNAs and 778 miRNAs. As described in more detail below, this was reduced to 400 mRNAs and 41 miRNAs (441 features). When training the single-layer 35 neuron neural network using 441 features, average accuracy for predicting molecular subtypes in the cross-validation testing set of the discovery cohort was 83%. After recursive feature elimination, a 31-feature signature consisting of 24 mRNAs and 7 miRNAs resulted in optimal model performance with an average accuracy of 96% across cross-validation testing sets. FIG. 6 exhibits model performance as a function of features included in the classifier, while Table 6 lists the specific mRNAs and miRNAs comprising the classifier.

TABLE 6

| Feature Included in Classifier | Feature Type |
|---|---|
| JAML | mRNA |
| PREX2 | mRNA |
| FAP | mRNA |
| MITF | mRNA |
| LDB2 | mRNA |
| LRRC8C | mRNA |
| DDR2 | mRNA |
| TSSC4 | mRNA |
| CRIPT | mRNA |
| TCIRG1 | mRNA |
| PKD2 | mRNA |
| ITPR1 | mRNA |
| ERF | mRNA |
| CFAP97 | mRNA |
| RARS2 | mRNA |
| PIK3CA | mRNA |
| ATAD1 | mRNA |
| CEBPZ | mRNA |
| RYK | mRNA |
| REST | mRNA |
| RIF1 | mRNA |
| USP34 | mRNA |
| INO80D | mRNA |
| RBMX | mRNA |
| MIR548X | miRNA |
| MIR21 | miRNA |
| MIR8072 | miRNA |
| MIR762 | miRNA |
| MIR92B.1 | miRNA |
| MIR7515 | miRNA |
| MIR30C.1 | miRNA |

### Classification of Molecular Subtypes in Validation Cohort

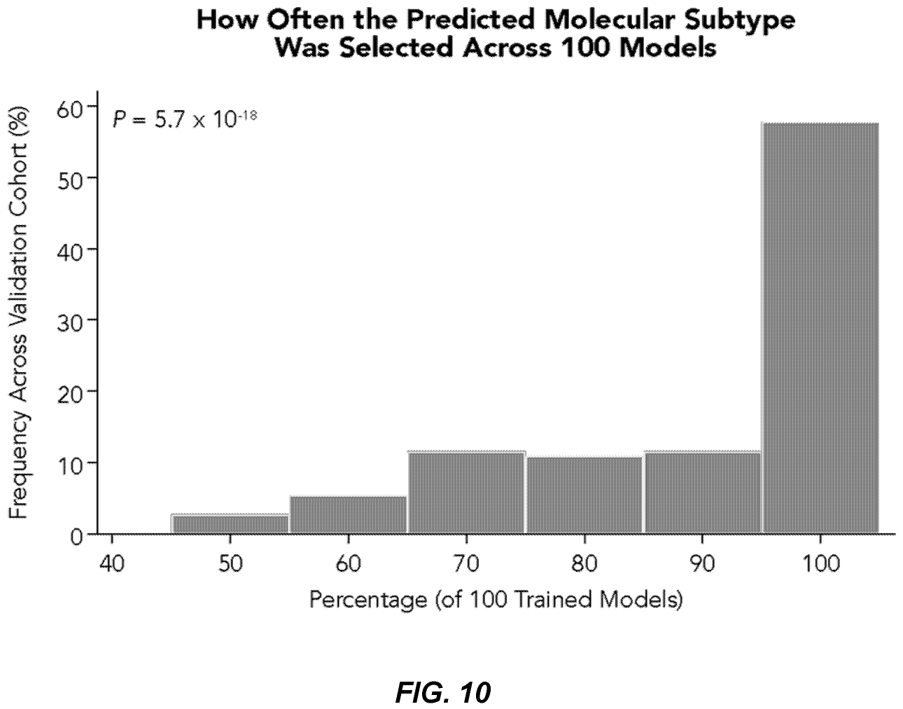

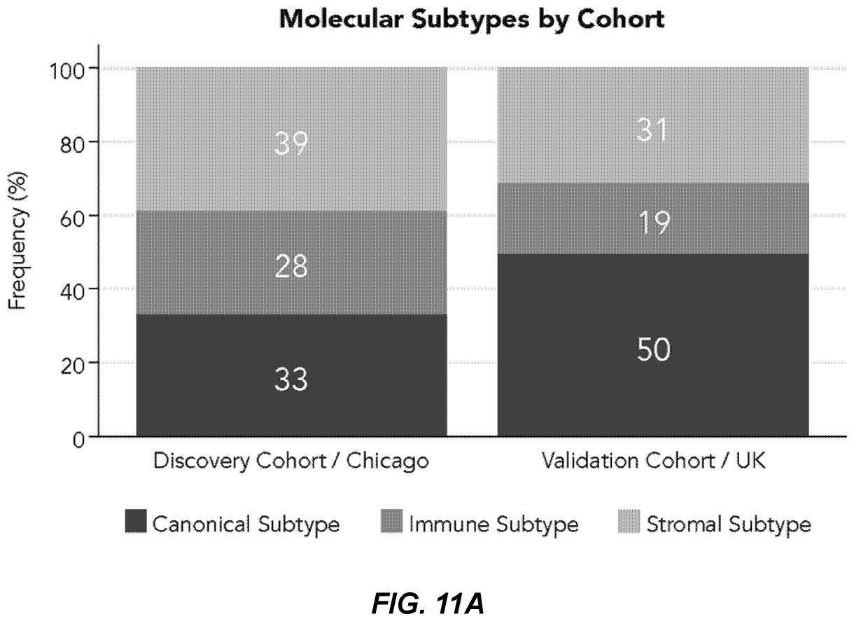

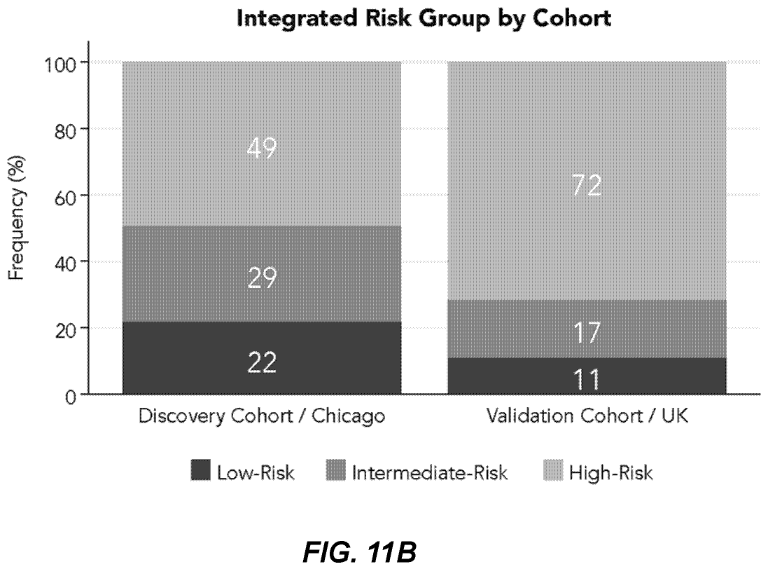

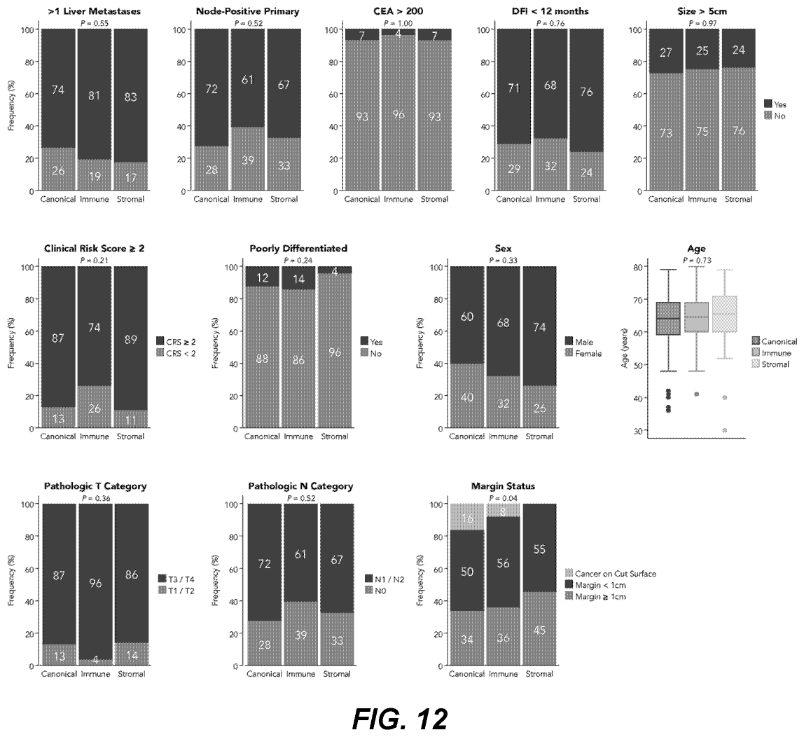

The molecular subtype for each liver metastasis in the validation cohort was determined by applying the neural network classifier using microarray data as input. Robustness and internal consistency of the classifier was supported by the strong concordance of predicted molecular subtypes across all 100 neural network models in this independent cohort (FIG. 10). Overall, the distributions of molecular subtypes in the discovery and validation cohorts were different (P=0.04 by Fisher's exact), with an increase in the canonical subtype (50% vs. 33%) in the validation cohort (FIG. 11A). Furthermore, the distribution of integrated clinical-molecular risk groups differed (P=0.007 by Fisher's exact), with increased high-risk patients (72% vs. 49%) in the validation cohort (FIG. 11B). Taken together, these findings demonstrated an increase in the frequency of adverse molecular subtypes and high-risk patients in the validation cohort.

Across molecular subtypes in the validation cohort, the incidence of positive margins (defined as cancer present on cut surface) was 16%, 8%, and 0% for canonical, immune, and stromal metastases, respectively (P=0.044). However, there was no difference in positive margin rate across molecular subtypes in the discovery cohort (P=0.70), suggesting this did not represent a true underlying relationship. There were no other differences in the clinical or pathological features included in the Clinical Risk Score, tumor and nodal staging, tumor differentiation, age, or sex in the validation cohort (Supplemental FIG. 4).⁴ These findings were consistent with the notion that molecular risk stratification largely provided additional prognostic information that was independent of the commonly used clinico-pathologic features.

### Molecular Phenotypes of Liver Metastases in the Validation Cohort

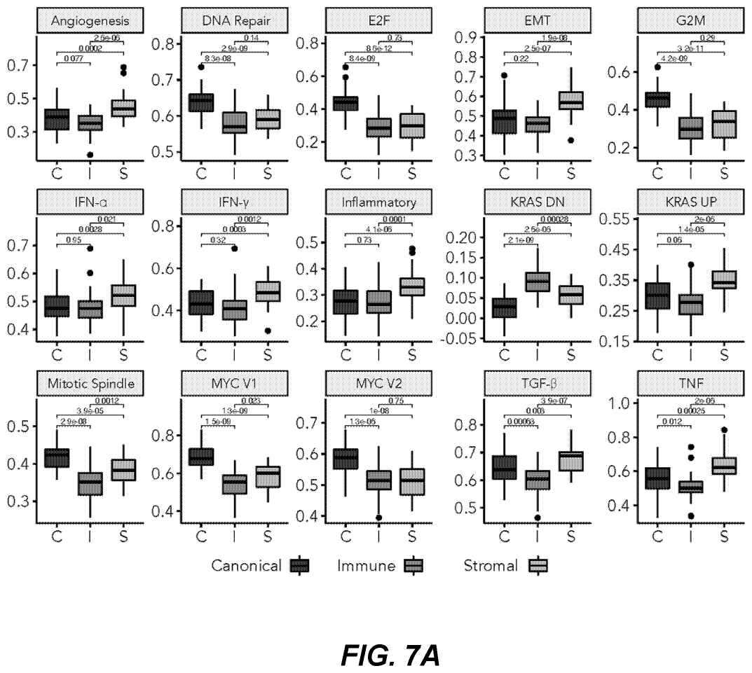

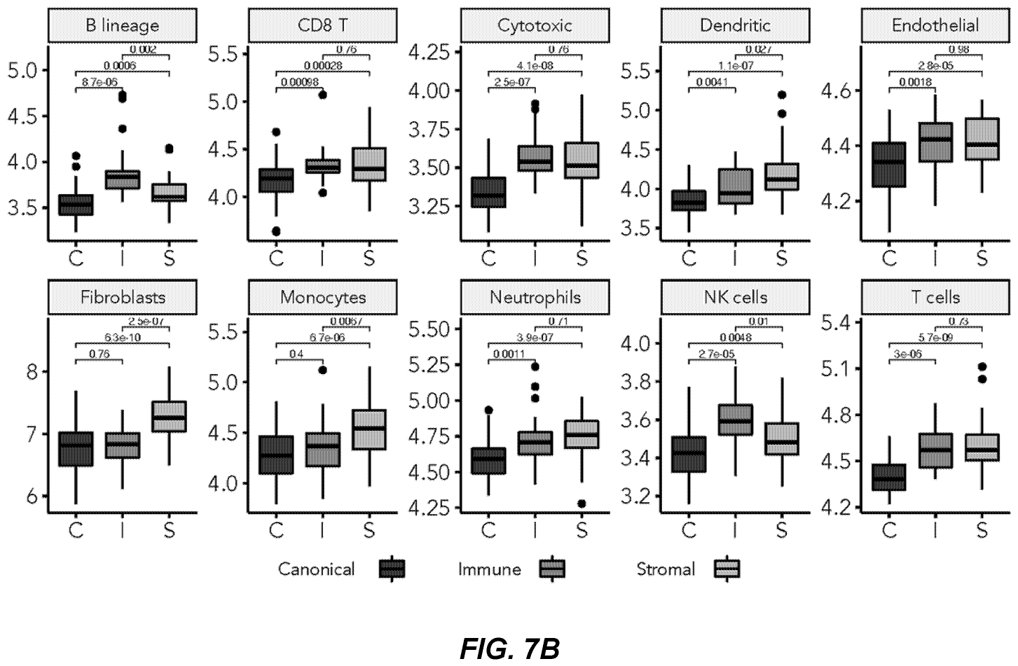

To corroborate the phenotype of each molecular subtype in the validation cohort, an ssGSEA analysis was performed (FIG. 7A). Consistent with the previous findings, the canonical subtype exhibited increased enrichment scores corresponding to DNA repair pathways, cell cycle regulation/proliferation (including E2F, G2M, mitotic spindle pathways), and MYC signaling. The stromal subtype demonstrated enrichment for epithelial-mesenchymal transition (EMT), angiogenesis, inflammatory response, and KRAS signaling. In addition, the immune subtype exhibited lower enrichment scores for KRAS signaling, angiogenesis, cell proliferation, and TGFβ signaling pathways.

Immune deconvolution analysis was performed in the validation cohort to evaluate the abundance of specific immune cells by molecular subtype (FIG. 7B). The majority of immune cells were decreased in the canonical subtype, whereas the immune subtype demonstrated enrichment for B cells, NK cells, CD8 T cells, and cytotoxic lymphocytes. By contrast, the stromal subtype exhibited depletion of B lymphocytes and NK cells and enrichment for fibroblast, monocytes, and myeloid dendritic cells in the context of CD8 T cells and cytotoxic lymphocytes. Though the presence of CD8 T and cytotoxic lymphocytes were similar between the immune and stromal subtypes, histological evaluation of the discovery cohort previously demonstrated that the spatial distribution of T cells in the tumor microenvironment was distinct.⁸ Immune metastases displayed dense band-like peritumoral and intratumoral infiltration of CD8 T lymphocytes, whereas stromal metastases exhibited significant fibrosis resulting in peritumorally restricted T lymphocytic infiltrate, which is consistent with increased fibroblasts in the stromal subtype. Collectively, these findings elucidated the distinct underlying molecular phenotypes associated with each of the subtypes.

### Clinical Outcomes for Discovery and Validation Cohorts

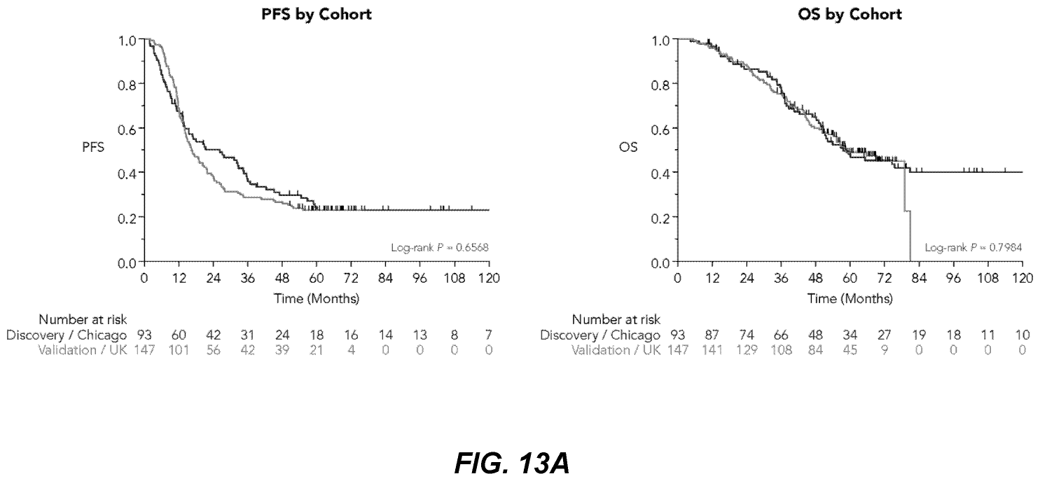

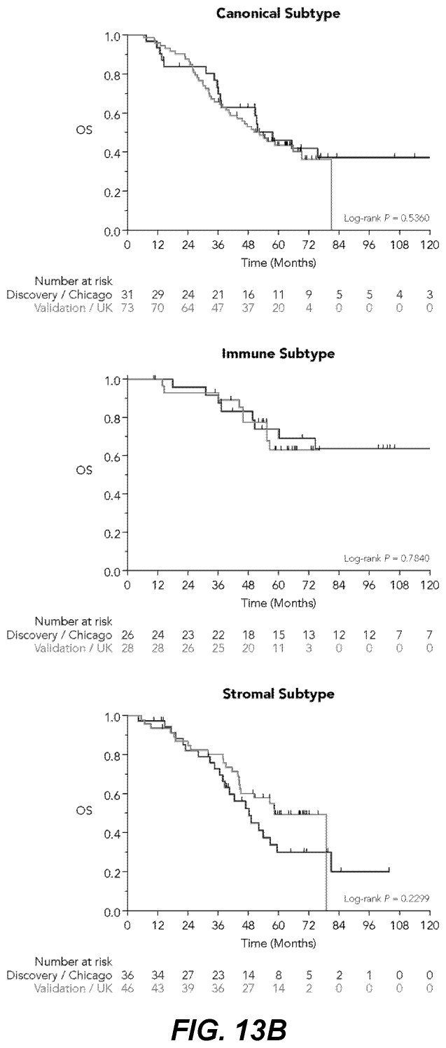

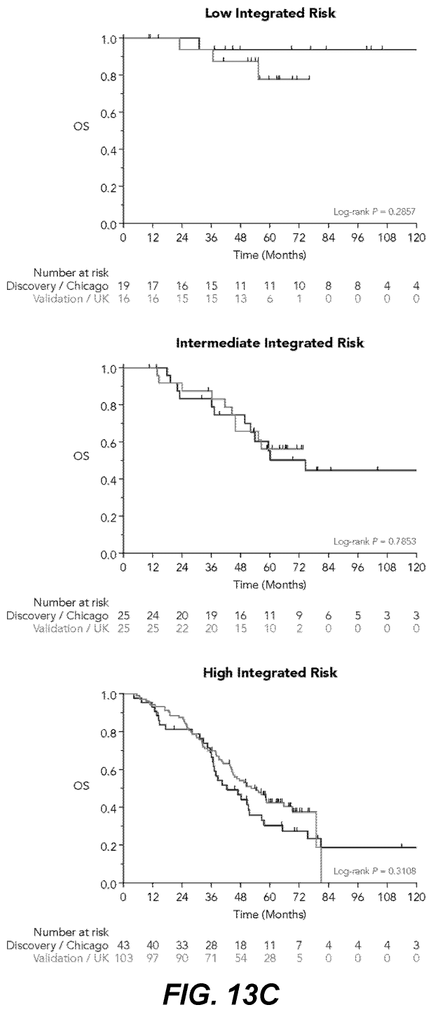

The overall PFS and OS were highly concordant between the discovery and validation cohorts (FIG. 13A). Specifically, in the discovery and validation cohorts the 5-year PFS was 24.3% and 23.0% and the 5-year OS was 48.2% and 49.0%, respectively. When split by molecular subtype, there were also no significant differences in OS between discovery and validation cohorts (FIG. 13B). Similarly, there were no differences in OS between discovery and validation cohorts when split by integrated clinical-molecular risk group (FIG. 13C). Collectively, these data demonstrated strong concordance in clinical outcomes across the two cohorts by molecular subtype and integrated clinical-molecular risk group.

### Prognostic Significance of Molecular Classifier in Validation Cohort

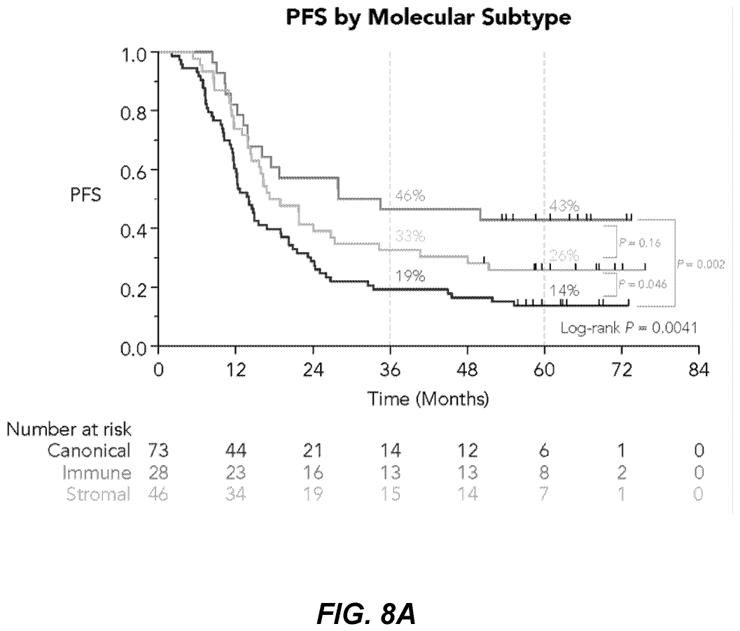

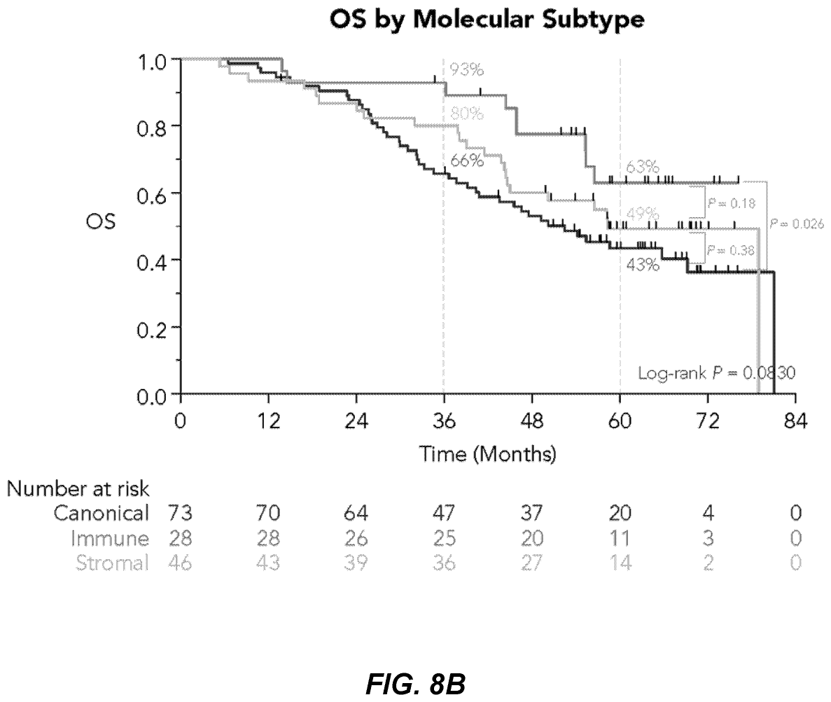

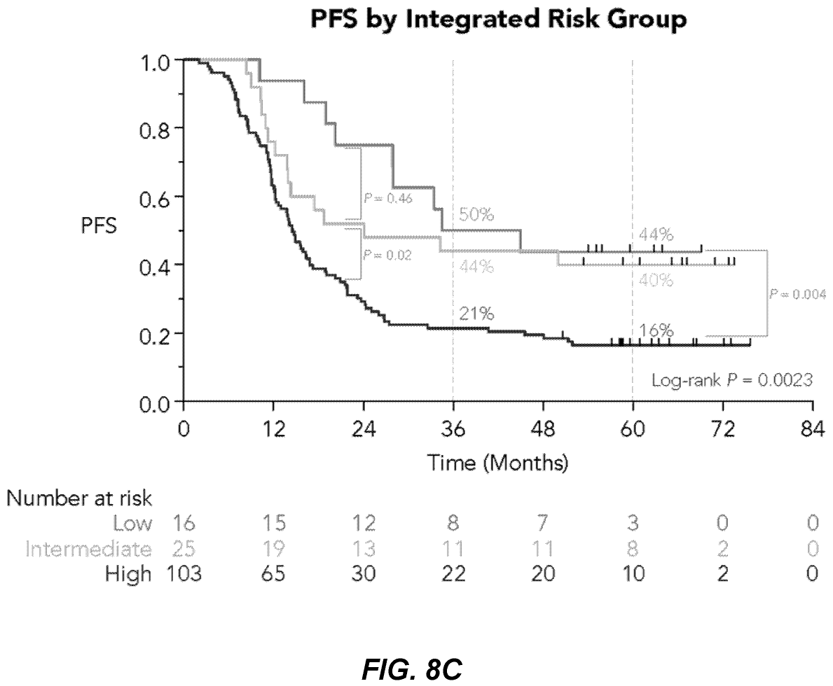

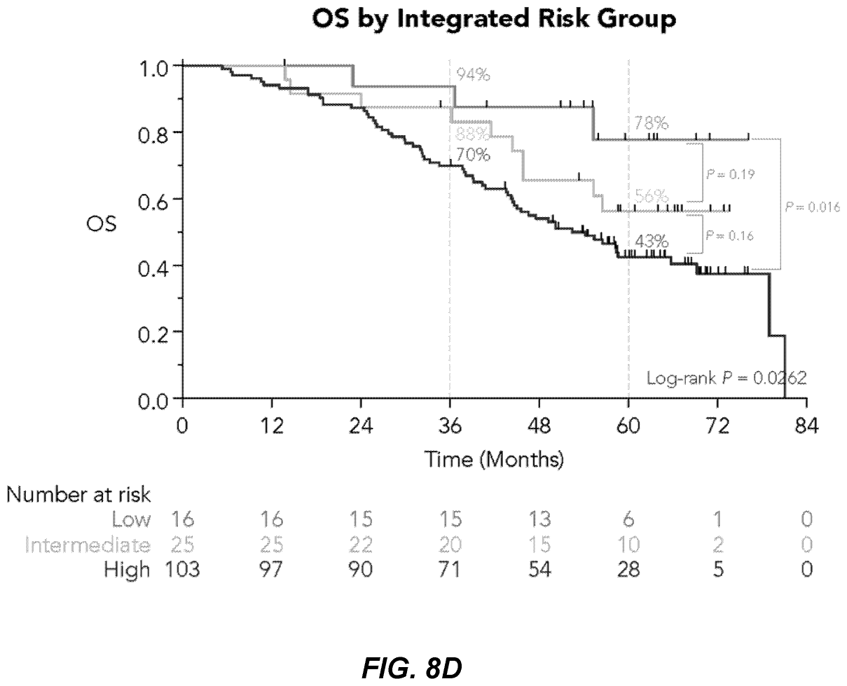

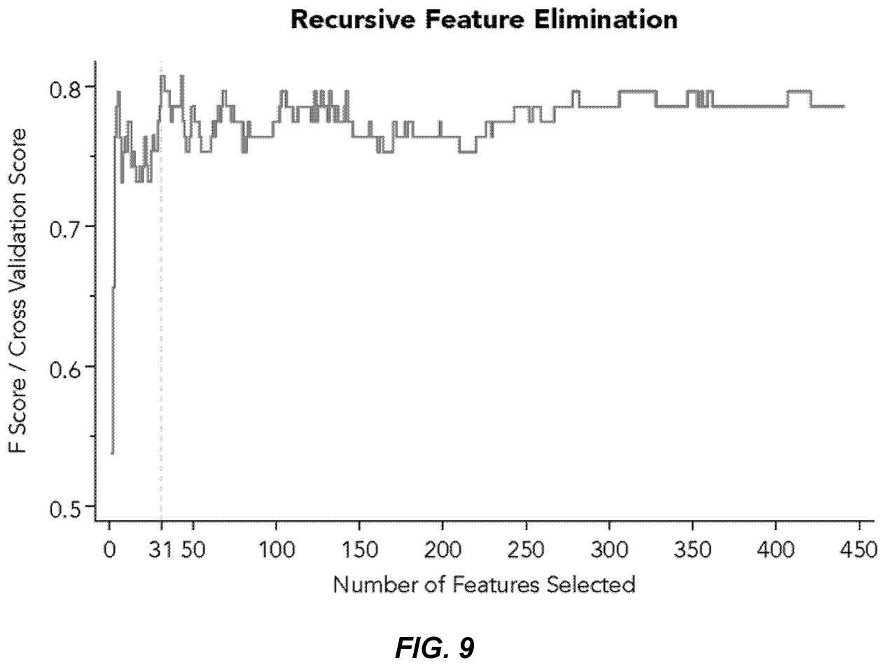

PFS and OS were analyzed in the validation cohort by molecular subtype of the liver metastasis and integrated clinical-molecular risk group to validate both as prognostic biomarkers. Using the Kaplan-Meier method, the immune subtype demonstrated the best PFS and OS as compared to canonical and stromal subtypes, consistent with previous findings (FIG. 8A). The 5-year PFS was 42.9% (95% CI, 24.6% to 60.0%), 13.7% (95% CI, 7.0% to 22.6%), and 25.9% (95% CI, 14.3% to 39·1%) for immune, canonical, and stromal subtypes, respectively. Differences in PFS were statistically significant across subtypes (log-rank P=0.004). There was a trend for a difference in OS by subtype alone (log-rank P=0.083, FIG. 8B). In this regard, the 5-year OS was 63.0% (95% CI, 40.3% to 79.0%), 43.4% (95% CI, 31.5% to 54.7%), and 49.4% (95% CI, 33.6% to 63.3%) for immune, canonical, and stromal subtypes, respectively. By pairwise comparison, this resulted in a statistically significant difference in OS between immune versus canonical/stromal subtypes (log-rank P=0.045).

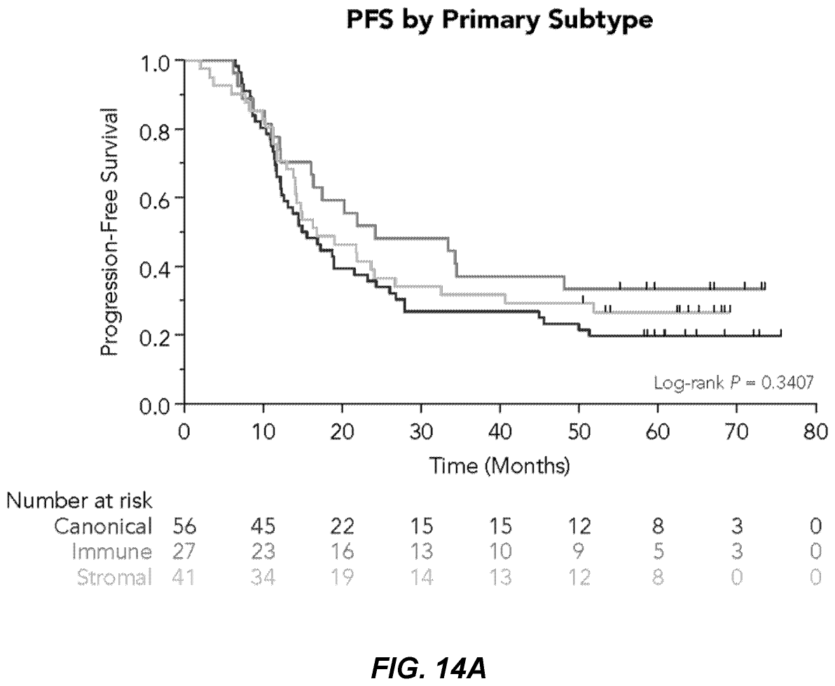

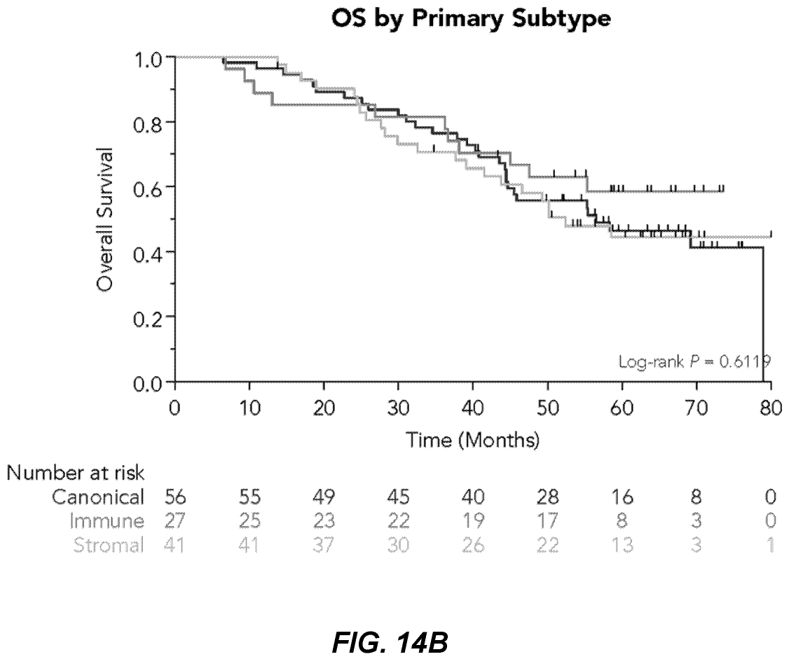

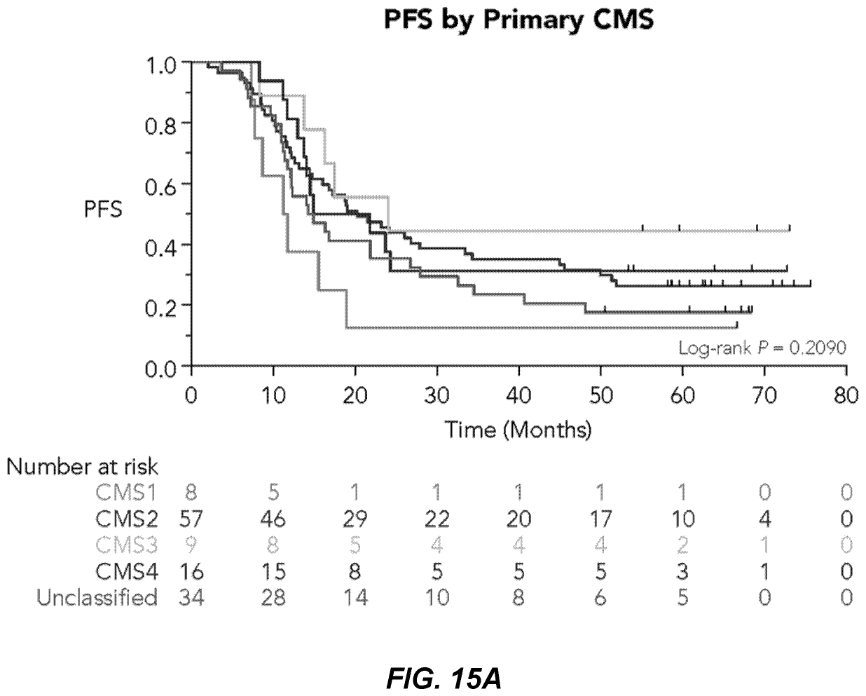

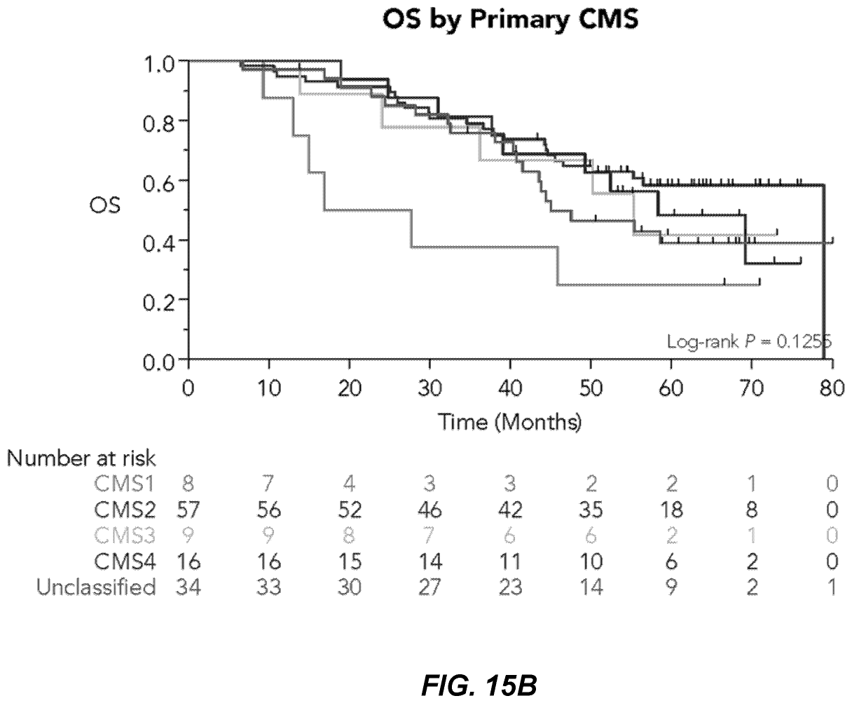

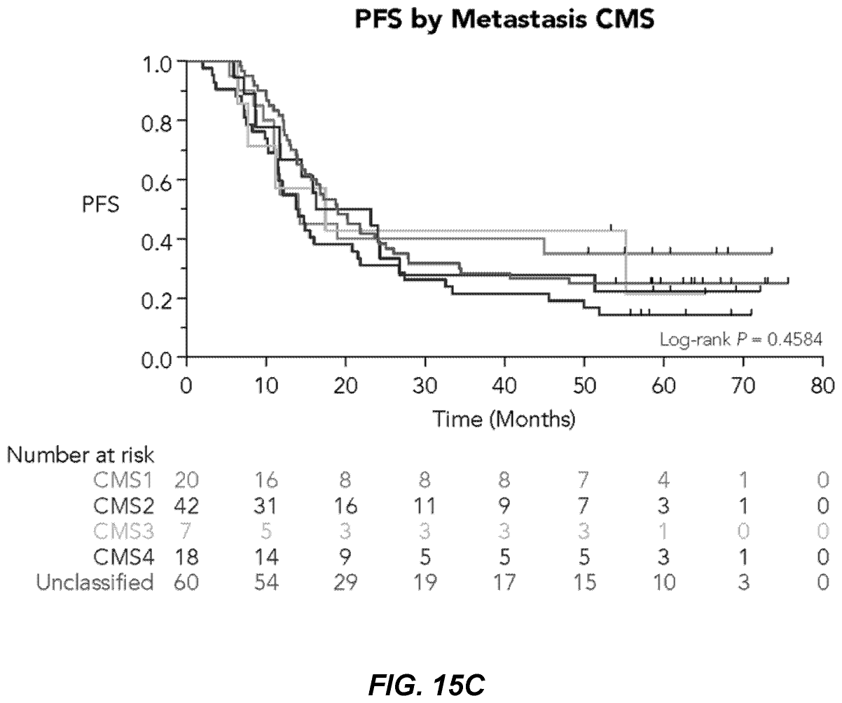

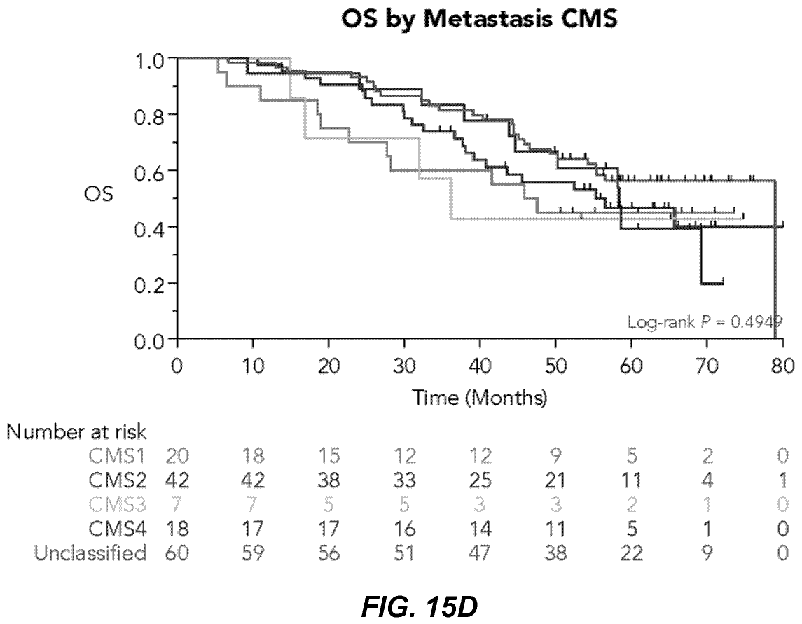

The neural network classifier was also applied to the primary tumor expression data to determine whether these subtypes were also discernable in primary tumors. There was no statistically significant association between predicted molecular subtypes in primary tumors and PFS or OS (FIGS. 14A-14B). When consensus molecular subtypes were determined for the primary tumors, there was no association between the CMS of the primary and the molecular subtype of the metastasis (Table 7).¹² Finally, neither the CMS subtype of the primary tumors nor the CMS subtype of the matched liver metastases were associated with PFS and OS, though 8 (6.5%) patients with primary tumor CMS1 exhibited a trend for worse OS, consistent with prior literature (FIGS. 15A-15D).¹⁴ Thus, the liver metastasis molecular subtypes were prognostic when applied to liver metastasis samples (with immune subtype demonstrating superior PFS and OS), while the liver metastasis molecular subtypes applied to the primary tumors and the CMS subtypes applied to either the primaries or metastases were not.

TABLE 7

| | | Molecular Subtype of Metastasis | | | |
|---|---|---|---|---|---|
| | | Canonical | Immune | Stromal | |
| | | Subtype | Subtype | Subtype | Total |
| | P = 0.37 | N = 59 | N = 27 | N = 38 | N = 124 |
| CMS of | CMS1 | 5 (62%) | 2 (25%) | 1 (12%) | 8 (100%) |
| Primary | CMS2 | 30 (53%) | 10 (18%) | 17 (30%) | 57 (100%) |
| | CMS3 | 3 (33%) | 4 (44%) | 2 (22%) | 9 (100%) |
| | CMS4 | 9 (56%) | 4 (25%) | 3 (19%) | 16 (100%) |
| | Unclassified | 12 (35%) | 7 (21%) | 15 (44%) | 34 (100%) |

### Validation of Integrated Clinical-Molecular Risk Grouping

By integrated clinical-molecular risk group, 5-year PFS was 43.8% (95% CI, 19.8% to 65.6%), 40.0% (95% CI, 21.3% to 58.1%), and 16.4% (95% CI, 10.0% to 24.2%) for the low-, intermediate-, and high-risk groups, respectively (log-rank P=0.0023, FIG. 8C). Both low-risk (log-rank P=0.004) and intermediate-risk (log-rank P=0.02) patients exhibited superior PFS to high-risk patients by pairwise comparison. This translated to a statistically significant difference in OS by integrated risk group (log-rank P=0.026). The 5-year OS was 77.8% (95% CI, 44.2% to 92.6%), 56.3% (95% CI, 33.8% to 73 -7%), and 42.5% (95% CI, 32.4% to 52.2%) for the low-, intermediate-, and high-risk groups, respectively (FIG. 8D).

Multivariable Cox proportional hazard models were computed in the validation cohort (Table 8). Cetuximab exhibited a detrimental effect on survival in the original New EPOC randomized trial; therefore, Table 8 also displays a model that included randomization to cetuximab. For PFS, the immune subtype demonstrated a HR of 0.43 and the stromal subtype demonstrated a HR of 0.61 (compared to canonical) when controlling for the Clinical Risk Score. There was also a trend for improved OS for the immune subtype (HR 0.51, P=0.065). Nonetheless, the integrated clinical-molecular risk score remained strongly associated with both PFS and OS. Though randomization to cetuximab was associated with worse survival in the model, it did not notably impact the prognostic effect size of the molecular subtypes or integrated risk groups. There were no significant interaction effects between molecular subtype and Clinical Risk Score, molecular subtype and cetuximab, and integrated risk group and cetuximab (P>0.05). Finally, the prognostic effect of the integrated clinical-molecular risk grouping persisted in a sensitivity analysis that included randomization to cetuximab, age, tumor differentiation, and margin status in the model (Table 9). In summary, integrated clinical-molecular risk stratification was highly prognostic in this independent validation cohort, defining a low-risk subgroup with an OS of 78% at 5 years.

TABLE 8

| Primary Cox Models | | | Cox Models Including Cetuximab Randomization | | |
|---|---|---|---|---|---|
| PFS by Molecular Subtype | | | PFS by Molecular Subtype | | |
| | Hazard Ratio | | | Hazard Ratio | |
| Variable | (95% CI) | P | Variable | (95% CI) | P |
| Molecular Subtype | | | Molecular Subtype | | |
| Canonical | Reference | | Canonical | Reference | |
| Immune | 0.43 (0.24 to 0.75) | 0.003 | Immune | 0.43 (0.24 to 0.75) | 0.003 |
| Stromal | 0.61 (0.40 to 0.94) | 0.023 | Stromal | 0.61 (0.40 to 0.93) | 0.022 |
| Clinical Risk Score | | | Clinical Risk Score | | |
| CRS < 2 | Reference | | CRS < 2 | Reference | |
| CRS ≥ 2 | 1.96 (1.09 to 3.53) | 0.024 | CRS ≥ 2 | 1.97 (1.10 to 3.55) | 0.023 |
| | | | Cetuximab | | |
| | | | No | Reference | |
| | | | Yes | 1.07 (0.73 to 1.56) | 0.73 |
| OS by Molecular Subtype | | | OS by Molecular Subtype | | |
| | Hazard Ratio | | | Hazard Ratio | |
| Variable | (95% CI) | P | Variable | (95% CI) | P |
| Molecular Subtype | | | Molecular Subtype | | |
| Canonical | Reference | | Canonical | Reference | |
| Immune | 0.51 (0.24 to 1.04) | 0.065 | Immune | 0.51 (0.24 to 1.05) | 0.067 |
| Stromal | 0.75 (0.45 to 1.26) | 0.28 | Stromal | 0.69 (0.41 to 1·16) | 0.16 |
| Clinical Risk Score | | | Clinical Risk Score | | |
| CRS < 2 | Reference | | CRS < 2 | Reference | |
| CRS ≥ 2 | 2.40 (0.96 to 5 .99) | 0.061 | CRS ≥ 2 | 2.60 (1.04 to 6.53) | 0.041 |
| | | | Cetuximab | | |
| | | | No | Reference | |
| | | | Yes | 1.66 (1.03 to 2.66) | 0.037 |
| PFS by Integrated Risk | | | PFS by Integrated Risk | | |
| | Hazard Ratio (95% | | | Hazard Ratio (95% | |
| Variable | CI) | P | Variable | CI) | P |
| Integrated Risk | | | Integrated Risk | | |
| Low | 0.38 (0.19 to 0.76) | 0.006 | Low | 0.38 (0.19 to 0.76) | 0.006 |
| Intermediate | 0.52 (0.30 to 0.91) | 0.021 | Intermediate | 0.52 (0.30 to 0.91) | 0.021 |
| High | Reference | | High | Reference | |
| | | | Cetuximab | | |
| | | | No | Reference | |
| | | | Yes | 1.01 (0.70 to 1.48) | 0.94 |
| OS by Integrated Risk | | | OS by Integrated Risk | | |
| | Hazard Ratio (95% | | | Hazard Ratio (95% | |
| Variable | CI) | P | Variable | CI) | P |
| Integrated Risk | | | Integrated Risk | | |
| Low | 0.26 (0.08 to 0.84) | 0.024 | Low | 0.25 (0.08 to 0.79) | 0.019 |
| Intermediate | 0.62 (0.32 to 1.21) | 0.16 | Intermediate | 0.64 (0.33 to 1.26) | 0.20 |
| High | Reference | | High | Reference | |
| | | | Cetuximab | | |
| | | | No | Reference | |
| | | | Yes | 1.57 (0.99 to 2.51) | 0.057 |

TABLE 9

| | | |
|---|---|---|
| | Hazard Ratio | |
| Variable | (95% CI) | P |

PFS by Molecular Subtype

| | | |
|---|---|---|
| Molecular Subtype | | |
| Canonical | Reference | |
| Immune | 0.41 (0.23 to 0.75) | 0.004 |
| Stromal | 0.65 (0.41 to 1.04) | 0.070 |
| Clinical Risk Score | | |
| CRS < 2 | Reference | |
| CRS ≥ 2 | 1.90 (1.03 to 3.48) | 0.039 |
| Cetuximab | | |
| No | Reference | |
| Yes | 1.00 (0.67 to 1.49) | 0.99 |
| Age (years) | 0.98 (0.97 to 1.00) | 0.13 |
| Tumor | | |
| Differentiation | | |
| Well/Moderate | Reference | |
| Poor | 1.38 (0.71 to 2.67) | 0.34 |
| Shortest Margin | | |
| Between Cancer and | | |
| Cut Surface | | |
| Margin ≥ 1 cm | Reference | |
| Margin < 1 cm | 1.17 (0.75 to 1.82) | 0.49 |
| No Margin | 1.75 (0.86 to 3.57) | 0.12 |
| (Cancer Visible on | | |
| Cut Surface) | | |

OS by Molecular Subtype

| | | |
|---|---|---|
| Molecular Subtype | | |
| Canonical | Reference | |
| Immune | 0.49 (0.23 to 1·07) | 0.076 |
| Stromal | 0.85 (0.48 to 1.49) | 0.56 |
| Clinical Risk Score | | |
| CRS < 2 | Reference | |
| CRS ≥ 2 | 2.20 (0.86 to 5.60) | 0.10 |
| Cetuximab | | |
| No | Reference | |
| Yes | 1.48 (0.91 to 2.42) | 0.12 |
| Age (years) | 1.00 (0.97 to 1.02) | 0.96 |
| Tumor | | |
| Differentiation | | |
| Well/Moderate | Reference | |
| Poor | 0.85 (0.36 to 2.01) | 0.71 |
| Shortest Margin | | |
| Between Cancer and | | |
| Cut Surface | | |
| Margin ≥ 1 cm | Reference | |
| Margin < 1 cm | 1.05 (0.61 to 1.81) | 0.87 |
| No Margin | 2.85 (1.30 to 6.23) | 0.009 |
| (Cancer Visible on | | |
| Cut Surface) | | |

PFS by Integrated Risk

| | | |
|---|---|---|
| Integrated Risk | | |
| Low | 0.40 (0.20 to 0.81) | 0.011 |
| Intermediate | 0.48 (0.27 to 0.86) | 0.014 |
| High | Reference | |
| Cetuximab | | |
| No | Reference | |
| Yes | 0.95 (0.63 to 1.41) | 0.78 |
| Age (years) | 0.98 (0.96 to 1.00) | 0.11 |
| Tumor | | |
| Differentiation | | |
| Well/Moderate | Reference | |
| Poor | 1.26 (0.65 to 2.45) | 0.49 |
| Shortest Margin | | |
| Between Cancer and | | |
| Cut Surface | | |
| Margin ≥ 1 cm | Reference | |
| Margin < 1 cm | 1.24 (0.80 to 1.92) | 0.33 |
| No Margin | 2.13 (1.08 to 4.21) | 0.029 |
| (Cancer Visible on | | |
| Cut Surface) | | |

OS by Integrated Risk

| | | |
|---|---|---|
| Integrated Risk | | |
| Low | 0.28 (0.09 to 0.92) | 0.035 |
| Intermediate | 0.59 (0.29 to 1.22) | 0.16 |
| High | Reference | |
| Cetuximab | | |
| No | Reference | |
| Yes | 1.46 (0.90 to 2.37) | 0.13 |
| Age (years) | 1.00 (0.97 to 1.03) | 0.99 |
| Tumor | | |
| Differentiation | | |
| Well/Moderate | Reference | |
| Poor | 0.86 (0.36 to 2.05) | 0.74 |
| Shortest Margin | | |
| Between Cancer and | | |
| Cut Surface | | |
| Margin ≥ 1 cm | Reference | |
| Margin < 1 cm | 1.07 (0.62 to 1.84) | 0.81 |
| No Margin | 3.02 (1.45 to 6.30) | 0.003 |
| (Cancer Visible on | | |
| Cut Surface) | | |

TABLE 10

| | | | Discovery Cohort/ | | Validation Cohort/ | | |
|---|---|---|---|---|---|---|---|
| | Total | | Chicago | | UK | | |
| | N = 240 | | N = 93 | | N = 147 | | P |
| Age, years, mean | 63.0 | (56.3-68.0) | 60.8 | (52.3-65.6) | 64.0 | (59.0-69.0) | <0.001 |
| (range) | | | | | | | |
| Sex | | | | | | | 0.22 |
| Female | 89 | (37.1%) | 39 | (41.9%) | 50 | (34.0%) | |
| Male | 151 | (62.9%) | 54 | (58.1%) | 97 | (66.0%) | |
| Clinical Risk Score | | | | | | | <0.001 |
| CRS < 2 | 53 | (22.1%) | 32 | (34.4%) | 21 | (14.3%) | |
| CRS ≥ 2 | 178 | (74.2%) | 55 | (59.1%) | 123 | (83.7%) | |
| Incomplete | 9 | (3.8%) | 6 | (6.5%) | 3 | (2.0%) | |
| Number of Liver | 151 | (63.7%) | 39 | (41.9%) | 112 | (77.8%) | <0.001 |
| Metastases > 1 | | | | | | | |
| Node-Positive Primary | 151 | (66.8%) | 55 | (64.0%) | 96 | (68.6%) | 0.56 |
| Pre-operative CEA > | 12 | (5.5%) | 3 | (3.9%) | 9 | (6.3%) | 0.55 |
| 200 | | | | | | | |
| Disease-Free Interval < | 157 | (65.4%) | 51 | (54.8%) | 106 | (72.1%) | 0.008 |
| 12 months | | | | | | | |
| Metastasis Size > 5 cm | 61 | (25.4%) | 23 | (24.7%) | 38 | (25.9%) | 0.88 |
| Shortest Margin | | | | | | | 0.17 |
| Between Cancer and Cut | | | | | | | |
| Surface | | | | | | | |
| Margin ≥ 1 cm | 76 | (31.7%) | 24 | (25.8%) | 52 | (35.4%) | |
| Margin < 1 cm | 120 | (50.0%) | 48 | (51.6%) | 72 | (49.0%) | |
| No Margin (Cancer | 27 | (11.2%) | 14 | (15.1%) | 13 | (8.8%) | |
| Visible on Cut | | | | | | | |
| Surface) | | | | | | | |
| Not Available* | 17 | (7.1%) | 7 | (7.5%) | 10 | (6.8%) | |

As disclosed herein, the inventors developed a novel 31-feature neural network classifier using gene expression data to robustly classify colorectal cancer liver metastases as one of three molecular subtypes: canonical, immune, and stromal. Utilizing only 24 mRNAs and 7 miRNAs, the classifier is highly concordant with a sophisticated clustering algorithm that leverages whole RNA sequencing and broad miRNA profiling. Furthermore, the molecular phenotype of these subtypes and their prognostic significance was validated in a large independent cohort from the multicenter New EPOC randomized, controlled phase III trial. The molecular subtypes independently add to clinical risk stratification for oncologic outcomes after hepatic resection and an integrated clinical-molecular risk grouping remains highly prognostic for survival.

The disclosed findings can contribute to improving the management of oligometastatic colorectal cancer liver metastases in several aspects. First, despite evidence for an oligometastatic paradigm in advanced colorectal cancer, few biomarkers exist to optimally balance the benefit of aggressive local therapy with systemic therapies.^{2,15,16} Integrated risk stratification incorporating the molecular subtype of the liver metastasis identifies patients with the greatest risk of relapse and thus, may help personalize peri-operative systemic therapy. Overall, this study presents a novel molecular classification system of the metastatic tumor in colorectal cancer. Though consensus molecular subtypes (CMS) exist for primary colorectal tumors, their prognostic utility is absent when applied to colorectal liver metastases. Furthermore, almost one third of colorectal cancer liver metastases are unclassifiable by CMS.⁸ Thus, it is crucial to molecularly stage the metastasis separately from the primary tumor. This is reinforced in this analysis as the molecular subtypes of the primary tumor were not associated with survival.

The identification of biologically distinct molecular subtypes with different clinical outcomes presents a potential opportunity to personalize therapy for colorectal cancer liver metastases. Though adjuvant chemotherapy is commonly administered after surgery for liver metastases, multiple randomized trials have not demonstrated a benefit in OS.²⁰⁻²³ The differential benefit of adjuvant systemic therapies (including cytotoxic chemotherapy, immunotherapy, anti-angiogenesis agents, or other targeted therapies) across molecular subtypes or integrated risk groups warrants further investigation in future trials. Thus, peri-operative systemic therapy may be prioritized for the subgroups most likely to benefit, such as those with chemosensitive disease or at higher risk of metastatic progression.

### Methods

### Study Design and Participants

Study results were reported following the Reporting Recommendations for Tumor Marker Prognostic Studies (REMARK) guidelines.¹¹ A neural network molecular classifier was trained in a retrospective discovery cohort consisting of 93 patients treated at The University of Chicago Medical Center (Chicago, IL) and NorthShore University Hospital (Evanston, IL). Patients with colorectal adenocarcinoma underwent hepatic resection for limited liver metastases that presented either synchronously or metachronously (typically 1-5 lesions involving one or both lobes). 98% of patients received standard-of-care peri-operative chemotherapy. An independent validation cohort consisted of 147 patients enrolled in the multicenter, randomized, controlled phase III New EPOC trial.^{9,10} In this trial, patients with operable colorectal cancer liver metastases (including those deemed suboptimally resectable or at high risk of positive resection margins) underwent hepatic resection with peri-operative chemotherapy (fluorouracil, oxaliplatin, irinotecan-based) with or without cetuximab. Patients were excluded if they were ineligible for chemotherapy or had extrahepatic distant metastases. Thus, both cohorts were similar, representing patients undergoing surgery with peri-operative systemic therapy for limited colorectal cancer liver metastases.

### Procedures

Specimen processing, training and application of the neural network classifier for liver metastasis molecular subtypes, and subsequent molecular analyses are outlined in detail in the Supplemental Appendix. For the discovery cohort, formalin-fixed paraffin-embedded (FFPE) specimens from hepatic resections underwent whole transcriptome RNA sequencing and miRNA profiling.⁸ For the validation cohort, as part of the S:CORT consortium, archival liver metastasis and primary tumor FFPE blocks from the New EPOC clinical trial underwent mRNA and miRNA profiling with microarray.^{9,10}

In the discovery cohort, a machine learning neural network classifier was trained to classify colorectal cancer liver metastases into one of three molecular subtypes (canonical, immune, and stromal) using mRNA and miRNA expression features (FIG. 6). In this cohort, the inventors previously defined molecular subtypes using the similarity network fusion (SNF) clustering algorithm, and these served as the reference standard for training the neural network classifier.⁸ The final classifier contained 24 mRNAs and 7 miRNAs.

For each patient in the validation cohort, the neural network classifier was applied to predict the molecular subtype of the corresponding liver metastasis. Of 110,425 total xCel microarray probesets, model input was limited to the probesets that corresponded to the 31 features (24 mRNAs and 7 miRNAs).

Molecular subtypes of the liver metastases were utilized for the primary statistical analyses, as the signature was developed in liver metastases. To investigate if the signature's prognostic performance was specific to application in liver metastases only, the molecular subtypes were also predicted for matched primary tumors. Consensus molecular subtypes (CMSs) of both the liver metastases and primary tumors were also determined to compare the prognostic performance of CMSs with the study's liver metastasis subtypes.¹²

Unlike the discovery cohort, no gold standard reference existed against which to compare the computed subtypes in the validation cohort. Therefore, to confirm that the neural network classifier accurately captured the expected molecular phenotype of the computed molecular subtypes within the validation cohort, single sample gene-set enrichment analysis (ssGSEA) and immune deconvolution were performed utilizing gene expression data for each liver metastasis.^{13,14}

### Outcomes and Statistical Analysis

Each patient and specimen in the discovery and validation cohorts was annotated with baseline demographic, clinical, and pathologic information. From baseline clinical and pathologic information, the Clinical Risk Score (CRS) was computed.⁴

As previously defined, an integrated clinical-molecular risk group was designated for each patient, combining the computed molecular subtype with high (≥2) or low (<2) CRS.⁸ Low-risk patients were defined as exhibiting an immune or canonical subtype with low CRS. Intermediate-risk patients were defined as demonstrating an immune subtype with high CRS or stromal subtype with low CRS. High-risk patients were defined as having a canonical or stromal subtype with high CRS.

PFS was defined as time to recurrence, progression, or death, and OS was defined as time to death. Time-to-event outcomes were measured from date of surgery in the discovery cohort and date of randomization on trial in the validation cohort. PFS and OS were analyzed using the Kaplan-Meier method and log-rank tests. Multivariable Cox proportional hazards models were generated to estimate the prognostic effect of the molecular subtypes and integrated risk groups in the validation cohort. Statistical analysis for ssGSEA enrichment scores and immune deconvolution features consisted of t-tests for pairwise comparison between subtypes. To correct for multiple comparisons, P values were adjusted by controlling the false discovery rate (FDR<0.05).

### Specimen Characteristics and Assay Methods

For the discovery cohort, formalin-fixed paraffin-embedded (FFPE) specimens from hepatic resections were histologically reviewed by an expert pathologist. Three spatially separated 2-mm punch biopsies of tumor-rich areas within each metastasis were obtained for each specimen. Nucleic acids were extracted using the RecoverAll Total Nucleic Acid Isolation Kit. RNA integrity and quantity was assessed using an Agilent 2100 Bioanalyzer. Ribosomal RNAs were removed using the Illumina Ribo-Zero rRNA Removal Kit. Reverse-stranded paired-end 75 base-pair sequencing libraries were constructed using Illumina Total RNA Stranded Kits. Subsequently, libraries were sequenced on a HiSEQ2500 machine. For miRNA expression profiling, 500 ng of total RNA was processed for biotin labeling and the biotin-labeled targets were hybridized to Affymetrix miRNA 4.0 Array Chips in an Affymetrix 640 hybridization oven. Arrays were washed and stained in an Affymetrix Fluidics Station 450 and the arrays were scanned using the Affymetrix GeneChip Scanner 3000 7G. CEL intensity files were generated using GCOS software.

For the validation cohort, as part of the S:CORT consortium, archival liver metastasis and primary tumor FFPE blocks from the New EPOC clinical trial were profiled.^{9,10} Briefly, tumor material was identified on an adjacent hematoxylin and eosin-stained slide for macrodissection. Total RNA was extracted from sequential 5-mm sections using the Roche High Pure FFPE Extraction Kit (Roche Life Sciences) and amplified using the NuGen Ovation FFPE Amplification System v3 (NuGen San Carlos). The amplified product was hybridized to the Almac Diagnostics XCEL array (Almac), a cDNA microarray-based technology optimized for archival FFPE tissue, and analyzed using the Affymetrix Genechip 3000 7G scanner (Affymetrix). Quality control metrics relating to monitor image quality, in vitro transcription, hybridization to the array, and RNA degradation were assessed prior to uploading to the S:CORT server, where further quality control was performed. Expression data was downloaded from a privately accessed cBioPortal repository from S:CORT. CEL files were processed using Affymetrix Array Power Tools (APT).

### Neural Network Classifier Training for Molecular Subtyping

In the discovery cohort, a machine learning neural network classifier was trained to classify colorectal cancer liver metastases into one of three molecular subtypes (canonical, immune, and stromal) using mRNA and miRNA expression features. The reference standard for training the neural network classifier were the molecular subtypes previously published using the similarity network fusion (SNF) clustering algorithm in the discovery cohort.⁸ Of importance, although molecular subtypes were ultimately associated with survival in the discovery set, the original SNF algorithm clustered tumors based only on molecular features and not survival outcomes.

For 93 patients in the discovery set, expression data was available for 17,162 mRNAs and 778 miRNAs. After principal component analysis (PCA), 400 mRNAs were selected based on having the highest PC1 and PC2 loadings. 41 miRNAs were also selected because they were present in both the discovery and validation expression datasets. Notably, the neural network classifier performed most accurately when utilizing both mRNA and miRNA features, with suboptimal accuracy when using either mRNA or miRNA alone. This is consistent with the original clustering-based approach to define molecular subtypes.⁸ Only the combination of mRNA and miRNA expression identified prognostic subgroups, while using mRNA or miRNA alone did not. The discovery set was split into 60% of the samples to train the model and 40% to test model accuracy. Using the standardized Z-score of the 441 features (400 mRNAs and 41 miRNAs) as input, a neural network containing a single hidden layer of 35 neurons was trained. In this way, 100 total neural networks were trained using 100 random 60% (training)/40% (testing) groupings of the discovery set to optimize the model performance.

In addition, to reduce the number of model input features while optimizing model accuracy, recursive feature elimination was performed, where input features that did not contribute significantly to the model accuracy were successively eliminated. Recursive feature elimination used a support vector machine (SVM) classifier to select the lowest number of features that maximized the F1 model score (which represents the harmonic mean of the precision/positive predictive value and recall/sensitivity of a test). 5-fold cross-validation was used. The final neural network model contained 24 mRNAs and 7 miRNAs. 100 neural networks were again trained using 100 random 60% (training)/40% (testing) splitting. Each model outputs the probability that a given sample corresponds to canonical, immune, or stromal subtypes. The subtype selected by each model was the subtype that had the highest probability. The overall subtype classification for each sample was the most frequent subtype chosen across the 100 neural network models.

### Application of the Molecular Subtype Classifier in Validation Cohort

In the validation cohort, xCel CEL files were quantitated, RMA background normalized, and log2 transformed using APT version 2.11.4. Of 110,425 total xCel probesets, model input was limited to the probesets that corresponded to the 31 features (24 mRNAs and 7 miRNAs). If multiple probes corresponded to a gene of interest, the probe with maximum mean expression was selected. Each feature value was normalized across all samples to produce Z-scores. For each specimen, these standardized Z-scores for each of the 31 features served as model input into the trained 100 neural network models. As in the discovery cohort, the subtype selected for each model was the subtype with the highest probability. The overall molecular subtype assigned for each sample in the validation cohort was the most frequent subtype chosen across the 100 models. The “predicted molecular subtype” in the validation cohort was referred to as the “molecular subtype” for simplicity.

Robustness and internal consistency of the classifier was evaluated. Because the overall predicted molecular subtype for each specimen was the most commonly predicted subtype of the 100 neural network models, concordance across each of the 100 models could be assessed. A truly random (i.e. non-predictive) classifier would be expected to have 33.3% concordance across the 100 models.

When computing the molecular subtype of a liver metastasis (utilized for the primary statistical analyses), gene expression data from the liver metastasis alone was utilized as model input. To investigate if the signature's prognostic performance was specific to application in metastases only, the molecular subtypes were also predicted for corresponding primary tumors. In this case, model input for each patient was limited to gene expression data for the primary tumor alone. To compare the prognostic performance of consensus molecular subtypes (CMSs) with the study's metastasis subtypes, CMSs of both the liver metastases and primary tumors were also determined.¹² Finally, an exploratory analysis assessing any relationship between CMS of the primary tumor and molecular subtype of the liver metastasis was performed utilizing Fisher's exact test.

### Single Sample Gene-Set Enrichment Analysis

To confirm that the neural network classifier accurately captured the molecular phenotype of the computed molecular subtypes within the validation cohort, a single sample gene-set enrichment analysis (ssGSEA) was performed using the EGSEA package in R and the Hallmark gene set database. For each gene feature, the probe with the maximum average signal was selected. A gene matrix was created with row names corresponding to the Entrez IDs and columns corresponding to the sample IDs. The EGSEA function egsea.ma was performed using the gene expression data and the algorithm method set to ssgsea. The ssgsea algorithm is an expansion of the GSEA algorithm.¹³ In brief, for each sample the gene expressions were rank-normalized and the Empirical Cumulative Distribution Function (ECDF) was calculated for each gene in the pathway as well as all the remaining genes. An enrichment score for a given pathway was then calculated by integrating the differences between the ECDFs.

### Immune Deconvolution

To additionally validate the molecular phenotype of predicted molecular subtypes in the validation cohort, immune deconvolution from the transcriptome was performed to estimate the presence of various immune cells in the tumor microenvironment. First, probeset level data was collapsed to gene level by taking the mean of the probesets. Then, absolute abundance of eight immune and two stromal features was generated with the R package, MCPcounter that provides a matrix table where each row corresponds to a feature and each column to a sample.

### REFERENCES

The following references, to the extent that they provide exemplary procedural or other details supplementary to those set forth herein, are specifically incorporated herein by reference.
1. Hellman S, Weichselbaum R R. Oligometastases. J Clin Oncol 1995; 13:8-10.
2. Weichselbaum R R, Hellman S. Oligometastases revisited. Nat Rev Clin Oncol 2011; 8:378-82.
3. Palma D A, Salama J K, Lo S S, et al. The oligometastatic state—separating truth from wishful thinking. Nat Rev Clin Oncol 2014; 11:549-57.
4. Treasure T. Oligometastatic cancer: an entity, a useful concept, or a therapeutic opportunity? J R Soc Med 2012; 105:242-6.
5. Mehta N, Mauer A M, Hellman S, et al. Analysis of further disease progression in metastatic non-small cell lung cancer: implications for locoregional treatment. Int J Oncol 2004; 25:1677-83.
6. Hong J C, Salama J K. The expanding role of stereotactic body radiation therapy in oligometastatic solid tumors: What do we know and where are we going? Cancer Treat Rev 2017; 52:22-32.
7. Tosoian J J, Gorin M A, Ross A E, Pienta K J, Tran P T, Schaeffer E M. Oligometastatic prostate cancer: definitions, clinical outcomes, and treatment considerations. Nat Rev Urol 2017; 14:15-25.
8. Loh J, Davis I D, Martin J M, Siva S. Extracranial oligometastatic renal cell carcinoma: current management and future directions. Future Oncol 2014; 10:761-74.
9. Fong Y, Fortner J, Sun R L, Brennan M F, Blumgart L H. Clinical score for predicting recurrence after hepatic resection for metastatic colorectal cancer: analysis of 1001 consecutive cases. Ann Surg 1999; 230:309-18; discussion 18-21.
10. Tomlinson J S, Jarnagin W R, DeMatteo R P, et al. Actual 10-year survival after resection of colorectal liver metastases defines cure. J Clin Oncol 2007; 25:4575-80.
11. Kadri S, Long B C, Mujacic I, et al. Clinical Validation of a Next-Generation Sequencing Genomic Oncology Panel via Cross-Platform Benchmarking against Established Amplicon Sequencing Assays. J Mol Diagn 2017; 19:43-56.
12. Mann C D, Metcalfe M S, Leopardi L N, Maddern G J. The clinical risk score: emerging as a reliable preoperative prognostic index in hepatectomy for colorectal metastases. Arch Surg 2004; 139:1168-72.
13. Ivanecz A, Potrc S, Horvat M, Jagric T, Gadzijev E. The validity of clinical risk score for patients undergoing liver resection for colorectal Hepatogastroenterology 2009; 56:1452-8.
14. Pitroda et al., “Integrated molecular subtyping defines a curable oligometastatic state in colorectal liver metastasis,” Nature Communications 9:1793 (2018).
15. PCT Publication WO2019/204576 to Pitroda et al.
16 Duda et al., 2001, Pattern Classification, Second Edition, John Wiley & Sons, Inc., New York.
17. Hastie et al., 2001, The Elements of Statistical Learning, Springer-Verlag, New York.

1. A method comprising measuring expression levels of one or more genes listed in Table 1 or one or more miRNAs listed in Table 2 in a sample comprising tissue from a metastasis from a primary cancer tumor.

2. The method of claim 1, wherein the metastasis is a liver metastasis.

3. The method of claim 1 or 2, wherein the primary cancer tumor is a colorectal cancer tumor.

4. The method of any one of claims 1 to 3, wherein the expression levels of at least 2, 3, 4, 5, 6, 7, 8, 9, 10, 11, 12, 13, 14, 15, 16, 17, 18, 19, 20, 21, 22, 23, or 24 of the genes listed in Table 1 are measured.

5. The method of any one of claims 1 to 4, wherein the expression levels of at least 2, 3, 4, 5, 6, or 7 of the miRNAs listed in Table 2 are measured.

6. The method of any one of claims 1 to 5, wherein the expression levels of one or more genes listed in Table 1 and one or more miRNAs listed in Table 2 are measured.

7. The method of any one of claims 1 to 6, wherein the expression levels of one or more genes listed in Table 1 or one or more miRNAs listed in Table 2 are excluded from being measured.

8. The method of any one of claims 1 to 5, wherein the expression levels of all 24 genes listed in Table 1 and all 7 miRNAs listed in Table 2 are measured.

9. The method of any one of claim 1 to 6 or 8, wherein no expression levels of genes or miRNAs are measured other than those listed in Table 1 and Table 2.

10. The method of any one of claims 1 to 9, wherein the expression levels of the one or more genes or one or more miRNAs are within a predetermined amount of a mean expression level in metastases of a cohort of patients having one of the following three metastatic phenotypes: canonical, immune, or stromal.

11. The method of claim 10, wherein the cohort of patients comprises a representative sample of patients having a canonical, immune, or stromal metastatic phenotype and comprises at least 50, 100, 150, 200, 250, 300, 350, 400, 450, or 500 patients.

12. The method of any one of claims 1 to 11, wherein the expression levels of the one or more genes or one or more miRNAs indicate that the metastasis has a canonical, immune, or stromal phenotype.

13. The method of any one of claims 1 to 12, wherein an expression signature of the one or more genes or one or more miRNAs matches an expression signature of a canonical, immune, or stromal metastatic phenotype.

14. The method of any one of claims 1 to 13, wherein the expression levels of one or more genes listed in Table 1 or one or more miRNAs listed in Table 2 deviate by more or less than a predetermined amount from the mean expression levels of the one or more genes or the one or more miRNAs in metastases of a cohort of metastatic colorectal cancer patients having a mean five-year overall survival expectation that is less than 60% or a mean five-year disease-free survival expectation that is less than 30%.

15. The method of any one of claims 1 to 14, further comprising calculating a clinical risk score for the patient.

16. The method of any one of claims 1 to 15, further comprising analyzing the expression levels of the one or more genes or miRNAs using a multi-layer neural network classification process that includes an input layer, one or more hidden layers, and an output layer.

17. The method of claim 16, wherein the expression levels of the one or more genes or miRNAs comprise the input layer.

18. The method of claim 16 or 17, wherein the output layer comprises a classification of the expression level data of the input layer as indicating a canonical, an immune, or a stromal metastatic phenotype.

19. The method of any one of claims 16 to 18, wherein the classification process comprises determining the probability that the metastasis has a canonical, immune, or stromal metastatic phenotype.

20. The method of claim 19, wherein the classification process comprises determining each of the three probabilities of the metastasis having a canonical, immune, and metastatic phenotype.

21. The method of any one of claims 16 to 19, wherein the neural network classification process comprises a first hidden layer and a second hidden layer.

22. The method of claim 21, wherein the first hidden layer has exactly 35 nodes and the second hidden layer has exactly 3 nodes.

23. The method of any one of claims 1 to 22, further comprising administering a cancer therapy to the patient.

24. The method of claim 23, wherein the cancer therapy comprises a local cancer therapy and does not comprise a systemic cancer therapy.

25. The method of claim 23, wherein the cancer comprises an immunotherapy.

26. The method of any one of claims 1 to 25, wherein measuring the expression levels of the mRNAs or miRNAs comprises performing PCR using RNA obtained from the sample as a template.

27. The method of any one of claims 1 to 26, wherein measuring the expression levels of the mRNAs or miRNAs comprises hybridizing DNA to a microarray.

28. A method of treating metastatic cancer in a patient, the method comprising administering to the patient a local cancer therapy without administering systemic cancer therapy, administering to the patient an immunotherapy, or administering to the patient cetuximab, wherein the patient has been determined to have a metastasis having expression levels of one or more genes listed in Table 1 or one and/or more miRNAs listed in Table 2 that indicate a canonical or immune metastatic phenotype based on a multi-layer neural network classification process.

29. The method of claim 28, wherein the multi-layer neural network classification process comprises an input layer, one or more hidden layers, and an output layer.

30. The method of claim 29, wherein the input layer comprises the expression levels of the one or more genes or miRNAs.

31. The method of claim 30, wherein the input layer comprises the expression levels of at least 2, 3, 4, 5, 6, 7, 8, 9, 10, 11, 12, 13, 14, 15, 16, 17, 18, 19, 20, 21, 22, 23, or 24 of the genes listed in Table 1 and at least 2, 3, 4, 5, 6, or 7 of the miRNAs listed in Table 2.

32. The method of any one of claims 28 to 31, wherein the output layer comprises a classification of the expression level data of the input layer as indicating a canonical, an immune, or a stromal metastatic phenotype.

33. A method of treating metastatic cancer in a patient, the method comprising administering to the patient a local cancer therapy without administering systemic cancer therapy or administering to the patient an immunotherapy or cetuximab, wherein the patient has been determined to have a metastasis having expression levels of one or more genes listed in Table 1 or one or more miRNAs listed in Table 2 that are within a predetermined amount of the mean expression level of the one or more genes or miRNAs in metastases of a cohort of metastatic cancer patients having a mean overall five-year survival expectation that is at least 60% or a mean five-year disease-free survival expectation that is at least 30%.

34. The method of claim 33, wherein the patient has been determined to have a metastasis having expression levels of at least 2, 3, 4, 5, 6, 7, 8, 9, 10, 11, 12, 13, 14, 15, 16, 17, 18, 19, 20, 21, 22, 23, or 24 of the genes listed in Table 1 and/or at least 2, 3, 4, 5, 6, or 7 of the miRNAs listed in Table 2 that are within a predetermined amount or within predetermined amounts of the mean expression levels of the one or more genes or miRNAs in metastases of a cohort of metastatic cancer patients having a mean overall five-year survival expectation that is at least 60% or a mean five-year disease-free survival expectation that is at least 30%.

35. The method of claim 34, wherein the expression levels of the one or more genes indicate a canonical or immune metastatic phenotype.

36. The method of any one of claims 33 to 35, wherein an expression signature of the one or more genes or one or more miRNAs matches an expression signature of a canonical or immune metastatic phenotype.

37. The method of any one of claims 33 to 36, wherein the expression levels of the one or more genes have been used as an input layer of a multi-layer neural network classification system.

38. A method of treating metastatic cancer in a patient, the method comprising administering to the patient a local cancer therapy without administering systemic cancer therapy, wherein the patient has been determined to have an mRNA and/or miRNA expression profile indicating canonical or immune metastatic phenotype, wherein the mRNA expression profile is determined by determining the expression of one or more genes listed in Table 1 and the miRNA expression profile is determined by determining the expression of one or more genes listed in Table 2.

39. The method of claim 39, wherein the expression of one or more genes listed in Table 1 and one or more miRNAs listed in Table 2 are used as the input layer of a multi-layer neural network classification process.

40. A method of treating cancer in a patient having a metastasis from a primary cancer tumor, the method comprising: administering to the patient an immune checkpoint therapy or administering to the patient a local cancer therapy without administering a systemic cancer therapy, wherein the patient has been identified based on expression levels of one or more mRNA and/or miRNA species in the metastasis as belonging to a group of metastatic cancer patients with one or more of the following characteristics:
(a) a mean five-year overall survival expectation of at least 60%;
(b) a mean five-year disease-free survival expectation of at least 30%;
(c) a likelihood of experiencing metastatic recurrence after hepatic resection that is lower than the likelihood for patients outside of the group;
(d) a canonical metastatic phenotype; and
(e) an immune metastatic phenotype;
wherein the one or more the one or more mRNA species comprise 1, 2, 3, 4, 5, 6, 7, 8, 9, 10, 11, 12, 13, 14, 15, 16, 17, 18, 19, 20, 21, 22, 23, or 24 of the genes listed in Table 1; and
wherein the one or more miRNA species comprise at least 1, 2, 3, 4, 5, 6, or 7 of the miRNAs listed in Table 2.

41. The method of claim 40, wherein the one or more mRNA species do not comprise transcripts of any genes other than those listed in Table 1, and the one or more miRNA species do not comprise any miRNAs other than the miRNAs listed in Table 2.

42. The method of claim 40 or 41, wherein the metastasis is a liver metastasis and the cancer is colorectal cancer.

43. A method of diagnosing a patient having a metastasis from a primary colorectal cancer tumor, the method comprising:
(a) determining expression levels in the metastasis of one or more of the genes listed in Table 1 or of one or more miRNAs listed in Table 2;
(b) identifying the patient as having a canonical metastatic phenotype, as having an immune metastatic phenotype, as being a responder to immune checkpoint cancer therapy, as having a five-year overall survival expectation of greater than 60%, or as having a five-year disease-free survival expectation of greater than 30% if the expression level of one or more of the genes or miRNAs is within a predetermined amount of a first reference expression level or deviates from a second reference expression level by a predetermined amount.

44. The method of claim 43, wherein the first reference expression level represents the mean expression level in metastases of a cohort of metastatic cancer patients having a canonical metastatic phenotype, having an immune metastatic phenotype, being a responders to immune checkpoint cancer therapy, having a five-year overall survival expectation of greater than 60%, and/or having a five-year disease-free survival expectation of greater than 30%.

45. The method of claim 43 or 44, wherein the second reference expression level represents the mean expression level in metastases of a cohort of metastatic cancer patients having a mean five-year overall survival expectation of less than 60%.

46. A method of diagnosing and treating a patient having a metastasis from a primary colorectal cancer tumor, the method comprising:
(a) measuring the expression of one or more genes or miRNAs in a sample from the metastasis;
(b) comparing the measured expression level of each gene or miRNA to a reference expression level for that gene or miRNA;
(c) identifying the metastasis as having a canonical, immune, or stromal phenotype based on the measured expression levels; and
(d) administering to the patient an appropriate therapy based on the type of metastasis identified in step (c).

47. The method of claim 46, wherein step (a) comprises measuring the expression of at least 1, 2, 3, 4, 5, 6, 7, 8, 9, 10, 11, 12, 13, 14, 15, 16, 17, 18, 19, 20, 21, 22, 23, or 24 of the genes listed in Table 1 and/or at least 1, 2, 3, 4, 5, 6, or 7 of the miRNAs listed in Table 2.

48. The method of claim 46 or 47, wherein step (b) comprises analyzing the expression level of each gene or miRNA using a multi-layer neural network classification system having an input layer, one or more hidden layers, and an output layer, wherein the input layer comprises the expression levels of the one or more genes or miRNAs and wherein the output layer comprises a classification of the expression level data of the input layer as indicating a canonical, an immune, or a stromal metastatic phenotype.

49. The method of any one of claims 46 to 48, wherein the appropriate therapy for a patient with a canonical-type metastasis comprises a DNA damaging chemotherapy, PARP inhibitor, angiogenesis inhibitor, or MYC inhibitor.

50. The method of any one of claims 46 to 48, wherein the appropriate therapy for a patient with an immune-type metastasis comprises cetuximab, immunotherapy, or a splicing inhibitor.

51. The method of any one of claims 46 to 48, wherein the appropriate therapy for a patient with a stromal-type metastasis comprises an angiogenesis inhibitor, KRAS inhibitor, or tumor stromal inhibitor, or excludes cetuximab.

52. A method comprising evaluating expression levels of one or more genes listed in table 1 and/or one or more miRNAs listed in Table 2 in a sample comprising tissue from a liver metastasis of a patient that has metastatic colorectal cancer to identify the patient as belonging to a first group of patients or a second group of patients, wherein said evaluating comprises using the expression levels as an input layer in a multi-layer neural network classification process and wherein:
(a) the first group has one or more of the following characteristics:
(i) a mean five-year overall survival expectation of at least 60%;
(ii) a mean five-year overall survival expectation that is higher than that for patients outside of the first group;
(iii) a likelihood of experiencing metastatic recurrence after hepatic resection that is lower than the likelihood for patients outside of the first group;
(iv) a likelihood of being successfully treated without systemic cancer treatments that is higher than the likelihood for patients outside of the first group;
(v) a likelihood of being successfully treated with immune checkpoint therapy that is higher than the likelihood for patients outside of the first group;
(vi) a mean five-year disease-free survival expectation of greater than 30%; and
(vii) a mean five-year disease-free survival expectation that is higher than that for patients outside of the first group; and
(b) the second group has one or more of the following characteristics:
(i) a mean five-year overall survival expectation of less than 60%;
(ii) a mean five-year overall survival expectation that is lower than that for patients outside of the second group;
(iii) a likelihood of experiencing metastatic recurrence after hepatic resection that is higher than for patients outside of the second group;
(iv) a likelihood of being successfully treated without systemic cancer treatments that is lower than the likelihood for patients outside of the second group;
(v) a likelihood of being successfully treated with immune checkpoint therapy that is lower than the likelihood for patients outside of the second group;
(vi) a likelihood of being successfully treated with DNA damaging cancer therapy that is higher than the likelihood for patients outside of the second group;
(vii) a mean five-year disease-free survival expectation of less than 30%; and
(viii) a mean five-year disease-free survival expectation that is lower than that for patients outside of the second group.

53. The method of claim 52, wherein the expression levels of the genes comprise expression levels of transcripts of at least 2, 3, 4, 5, 6, 7, 8, 9, 10, 11, 12, 13, 14, 15, 16, 17, 18, 19, 20, 21, 22, 23, or 24 of the genes listed in Table 1.

54. The method of claim 52 or 53, wherein the expression levels of the miRNA species comprise expression levels of at least 2, 3, 4, 5, 6, or 7 of the miRNAs listed in Table 2.

55. The method of any one of claims 52 to 54, wherein the patient is identified as belonging to the first group of patients if the neural network classification process indicates that the metastasis has a canonical or immune phenotype.

56. The method of any one of claims 52 to 55, wherein the patient is identified as belonging to the second group of patients if the neural network classification process indicates that the metastasis has a stromal phenotype.

57. The method of any one of claims 52 to 56, further comprising administering an immune checkpoint therapy or cetuximab to a patient identified as belonging to the first group.

58. The method of any one of claims 52 to 57, further comprising treating a patient identified as belonging to the first group with local treatment of liver metastases unaccompanied by systemic cancer treatment.

59. The method of any one of claims 52 to 56, further comprising administering a DNA damaging cancer therapy to a patient identified as belonging to the second group of patients.

60. A method of diagnosing and treating a patient having a metastasis from a primary colorectal cancer tumor, the method comprising:
(a) measuring the expression of one or more genes listed in Table 1 or miRNAs listed in Table 2 in a sample from the metastasis;
(b) identifying the metastasis as having a canonical, immune, or stromal phenotype based on the measured expression levels using a neural network classification system; and
(d) administering to the patient an appropriate therapy based on the type of metastasis identified in step (c).

61. The method of claim 60, wherein step (a) comprises measuring the expression of at least 2, 3, 4, 5, 6, 7, 8, 9, 10, 11, 12, 13, 14, 15, 16, 17, 18, 19, 20, 21, 22, 23, or 24 of the genes listed in Table 1 and/or at least 2, 3, 4, 5, 6, or 7 of the miRNAs listed in Table 2.

62. The method of claim 60 or 61, wherein the appropriate therapy for a patient with a canonical-type metastasis comprises a DNA damaging chemotherapy, PARP inhibitor, angiogenesis inhibitor, or MYC inhibitor.

63. The method of claim 60 or 61, wherein the appropriate therapy for a patient with an immune-type metastasis comprises cetuximab, immunotherapy, or a splicing inhibitor.

64. The method of claim 60 or 61, wherein the appropriate therapy for a patient with a stromal-type metastasis comprises an angiogenesis inhibitor, KRAS inhibitor, or tumor stromal inhibitor, or excludes cetuximab.

65. A method of treating a patient having metastatic colorectal cancer, the method comprising administering cetuximab to a patient who has been tested and found to have liver metastases of an immune molecular subtype.

66. The method of claim 65, wherein the test comprises analyzing the expression levels of transcripts of at least 1, 2, 3, 4, 5, 6, 7, 8, 9, 10, 11, 12, 13, 14, 15, 16, 17, 18, 19, 20, 21, 22, 23, or 24 of the genes listed in Table 1 and at least 1, 2, 3, 4, 5, 6, or 7 of the miRNAs listed in Table 2.

67. The method of claim 66, wherein the expression levels of the genes and miRNAs are analyzed using a neural network classification process.

68. The method of claim 67, wherein the input into the neural network classification process consists of the 24 genes listed in Table 1 and the 7 miRNAs listed in Table 2.

69. A method of treating a patient having metastatic colorectal cancer, the method comprising administering a local cancer therapy unaccompanied by systemic cancer therapy to a patient who has been tested and found to have liver metastases of a canonical or immune molecular subtype, wherein the test comprises analyzing the expression levels of transcripts of one or more genes listed in Table 1 and one or more miRNAs listed in Table 2 using a neural network classification process.

70. The method of claim 69, wherein the input into the neural network classification process includes only genes listed in Table 1 and only miRNAs listed in Table 2.

71. The method of claim 70, wherein the input into the neural network classification process consists of the 24 genes listed in Table 1 and the 7 miRNAs listed in Table 2.

72. A method of diagnosing a patient having a liver metastasis from a primary colorectal cancer tumor, the method comprising inputting the expression levels in the metastasis of one or more of the genes listed on Table 1 and one or more of the miRNAs listed in Table 2 into a classifier that has been trained to recognize an expression signature of a canonical, immune, and/or stromal metastatic molecular subtype.

73. The method of claim 72, wherein the classifier has been trained using a neural network machine learning process.

74. The method of claim 72 or 73, wherein the expression levels of all 24 of the genes listed on Table 1 and all 7 of the miRNAs listed on Table 1 are inputted into the classifier.

75. The method of claim 74, wherein no other expression levels are inputted into the classifier.

76. A method of treating metastatic colorectal cancer in a patient, the method comprising administering to the patient a local cancer therapy unaccompanied by systemic cancer therapy, wherein the patient has been identified as having metastases of a canonical or immune subtype by a classifier that analyzed expression levels in a metastasis tissue sample from the patient of one or more of the genes listed in Table 1 and one or more of the miRNAs listed in Table 2, wherein the classifier was configured to recognize an expression signature of a canonical or immune subtype based on the expression levels.

77. The method of claim 76, wherein the only metastasis expression levels analyzed by the classifier are the 24 genes listed in Table 1 and the 7 miRNAs listed in Table 2.

78. The method of claim 76 or 77, wherein the classifier has been trained using a neural network machine learning process.
